(19) United States
(12) Patent Application Publication
(10) Pub. No.: US 2018/0344745 A1
(43) Pub. Date: Dec. 6, 2018

(54) TREATMENT OF DEVELOPMENTAL DISORDERS WITH IMIDAZOLONE DERIVATIVES

(71) Applicant: Ovid Therapeutics Inc., New York, NY (US)

(72) Inventor: Matthew DURING, Weston, CT (US)

(21) Appl. No.: 15/995,292

(22) Filed: Jun. 1, 2018

Related U.S. Application Data
(60) Provisional application No. 62/513,639, filed on Jun. 1, 2017.

(51) Int. Cl. A61K 31/5377; A61K 31/454; A61P 25/28; A61P 25/08

(52) U.S. Cl. CPC A61K 31/5377; A61P 25/08; A61P 25/28; A61K 31/454

(57) ABSTRACT

Methods of treating developmental disorders such as Angelman syndrome, Fragile X syndrome, Fragile X-associated tremor/ataxia syndrome (FXTAS), Autistic Spectrum Disorder, Autism, Asperger's syndrome, pervasive developmental disorder, Childhood Disintegrative Disorder, Rett syndrome, Landau-Kleffner Syndrome, Prader-Willi Syndrome, Tardive Dyskinesia, a seizure disorder and/or Williams Syndrome with a compound of formula 1 or a pharmaceutically acceptable salt thereof,

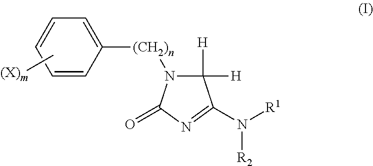

wherein X, R¹, R², m and n have the meanings set forth in the specification, are provided. The methods provide therapeutic compositions that may be used to improve one or more symptoms of the developmental disorder.

### CROSS REFERENCE TO RELATED APPLICATIONS

This application claims benefit of and priority to U.S. Provisional Application No. 62/513,639, filed Jun. 1, 2017, which is incorporated herein by reference in its entirety.

### TECHNICAL FIELD

Methods of treating developmental disorders with imidazolone derivatives, or a pharmaceutically acceptable salt thereof are provided.

### BACKGROUND

Imidazolone derivatives have been utilized as anticonvulsants and as antiepileptic drugs (AED). See, e.g., PCT Publication WO1997009314. Imepitoin (1-(4-chlorophenyl)-4-morpholino-imidazolin-2-one) was initially developed as a new AED for humans, but development for humans was terminated based on variable pharmacokinetics. For example, imepitoin is more rapidly eliminated in smokers than in non-smokers. See, Rundfeld and Löscher, CNS Drugs (2014) 28:29-43. Imepitoin potentiates the amplitude of γ-aminobutyric acid (GABA)-evoked currents by acting at the benzodiazepine (BZD) recognition site of the GABA_{A} receptor. In contrast to clinically used BZD ligands, imepitoin acts as a low-affinity partial agonist with low intrinsic activity. See, Loscher et al, Epilepsia, (2004) 45:10, 1228-1239. Imidazolone derivatives have been also been explored as anxiolytics, e.g., ELB139 (1-(4-chlorophenyl)-4-piperidinoimidazolin-2-one). See, e.g., U.S. Pat. No. 5,994,347. ELB139 has similar effects to BZT drugs, but is structurally distinct and may be classified as a nonbenzodiazepine anxiolytic. ELB-139 is a subtype-selective partial agonist at GABA_{A} receptors, with highest affinity for the α3 subtype, but highest efficacy at α1 and α2. Rabe et al., Neuropharmacology, (2007) 52 (3): 796-801.

Developmental disorders include Autistic Spectrum Disorder, pervasive developmental disorder, Autism, Angelman syndrome, Fragile X syndrome, Fragile X-associated tremor/ataxia syndrome (FXTAS), Rett syndrome, Asperger's syndrome, Childhood Disintegrative Disorder, Attention-deficit/hyperactivity disorder (ADHD), Landau-Kleffner Syndrome, Prader-Willi Syndrome, Rasmussen's syndrome, Dravet syndrome, Tardive Dyskinesia, and/or Williams Syndrome. Developmental disorders may encompass a seizure disorder such as epilepsy, epilepsy with generalized tonic-clonic seizures, epilepsy with myoclonic absences, frontal lobe epilepsy, temporal lobe epilepsy, infantile spasms (West syndrome), childhood absence epilepsy, juvenile myoclonic epilepsy (JME), vaccine-related encephalopathy, intractable childhood epilepsy (ICE), essential tremor, acute repetitive seizures, benign rolandic epilepsy, status epilepticus, refractory status epilepticus, super-refractory status epilepticus (SRSE), PCDH19 pediatric epilepsy, increased seizure activity (also called serial or cluster seizures), or breakthrough seizures. Seizure disorders may be associated with a sodium channel protein type 1 subunit alpha (Scn1a)-related disorder.

Treatments for developmental disorders such as Autistic Spectrum Disorder, Rett syndrome, Angelman syndrome, Fragile X syndrome, and Fragile X-associated tremor/ataxia syndrome are limited. Angelman syndrome is a neurodevelopmental disorder caused by loss of function of the UBE3A gene encoding a ubiquitin E3 ligase. Motor dysfunction is a characteristic feature of Angelman syndrome, but neither the mechanisms of action nor effective therapeutic strategies have yet been elucidated. Symptoms of Angelman syndrome may include developmental delays, including no crawling or babbling at 6 to 12 months, intellectual disability, no speech or minimal speech, frequent smiling and laughter, happy, excitable personality, seizures, usually beginning between 2 and 3 years of age, unusual behaviors, such as hand flapping and arms uplifted while walking, tongue thrusting, and stiff or jerky movements. Difficulty coordinating sucking and swallowing may cause feeding problems in infants. Abnormal sleep-wake patterns may be present. People diagnosed with Angelman syndrome may need less sleep than most people.

Fragile X syndrome may be the most common genetic cause of intellectual disability and the most common single-gene cause of autism. It is caused by mutations on the fragile X mental retardation gene (FMR1) and lack of fragile X mental retardation protein, which in turn, leads to decreased inhibition of translation of many synaptic proteins. The main efforts have focused on metabotropic glutamate receptor (mGluR) targeted treatments; however, investigation on the gamma-aminobutyric acid (GABA) system and its potential as a targeted treatment is less emphasized. The fragile X mouse models (Fmr1-knock out) show decreased GABA subunit receptors, decreased synthesis of GABA, increased catabolism of GABA, and overall decreased GABAergic input in many regions of the brain. These symptoms are also observed in individuals with autism and other neurodevelopmental disorders, therefore the targeted treatments for Fragile X syndrome are leading the way in the treatment of other neurodevelopmental syndromes and autism. Potential GABAergic treatments, such as riluzole, gaboxadol, tiagabine, and vigabatrin have been discussed. However, further studies are needed to determine the safety and efficacy of GABAergic treatments for Fragile X syndrome.

Fragile X-associated tremor/ataxia syndrome (FXTAS) is a late-onset disorder, usually occurring after age 50. Mutations in the FMR1 gene increase the risk of developing FXTAS. The mutation relates to a DNA segment known as a CGG triplet repeat which is expanded within the FMR1 gene. Normally, this DNA segment is repeated from 5 to about 40 times. In people with FXTAS the CGG segment may be repeated 55 to 200 times. This mutation is known as an FMR1 gene premutation. An expansion of more than 200 repeats, a full mutation, causes Fragile X syndrome discussed above. FXTAS is typically characterized by problems with movement and thinking ability (cognition). FXTAS signs and symptoms usually worsen with age. Affected individuals have areas of damage in the cerebellum, the area of the brain that controls movement. Characteristic features of FXTAS are intention tremor, which is trembling or shaking of a limb when trying to perform a voluntary movement such as reaching for an object, and problems with coordination and balance (ataxia). Many affected individuals develop other movement problems, such as parkinsonism, which includes tremors when not moving (resting tremor), rigidity, and unusually slow movement (bradykinesia). In addition, affected individuals may have reduced sensation, numbness or tingling, pain, or muscle weakness in the lower limbs, and inability to control the bladder or bowel. Other symptoms may include chronic pain syndromes, such as fibromyalgia and chronic migraine, hypothyroidism, hypertension, insomnia, sleep apnea, vertigo, olfactory dysfunction, and hearing loss. People with FXTAS commonly have cognitive disabilities such as short-term memory loss and loss of executive function, which is the ability to plan and implement actions and develop problem-solving strategies. Loss of this function impairs skills such as impulse control, self-monitoring, focusing attention appropriately, and cognitive flexibility. Many people with FXTAS experience psychiatric symptoms such as anxiety, depression, moodiness, or irritability.

There is currently no targeted therapeutic intervention that can arrest or reverse the pathogenesis of FXTAS. However a number of treatment approaches of potential symptomatic benefit have been suggested. Primidone, beta-blockers such as propanolol, topiramate, carbidopa/levodopa, and benzodiazepines have been suggested to control tremors associated with FXTAS; botulinum toxin for involuntary muscle activities, such as dystonia and spasticity; carbidopa/levodopa, amantadine and buspirone for ataxia; cholinesterase inhibitors such as donepezil, and memantine (an NMDA antagonist) for cognitive deficits and dementia; and antidepressants and antipsychotics for psychiatric symptoms. See, e.g., Hagerman, et al., Clin Intery Aging. 2008 June; 3(2): 251-262.

Rett syndrome is a neurodevelopmenal disorder that typically affects girls. It is characterized by normal early growth and development followed by a slowing of development, loss of purposeful use of the hands, distinctive hand movements, slowed brain and head growth, problems with walking, seizures, and intellectual disability. Nearly all cases of Rett syndrome are caused by a mutation in the methyl CpG binding protein 2, or MECP2 gene. The MECP2 gene contains instructions for the synthesis of methyl cytosine binding protein 2 (MeCP2), which is utilized in brain development and acts as one of the many biochemical switches that can either increase or decrease gene expression. The main diagnostic criteria or symptoms include partial or complete loss of acquired purposeful hand skills, partial or complete loss of acquired spoken language, repetitive hand movements (such has hand wringing or squeezing, clapping or rubbing), and gait abnormalities, including toe-walking or an unsteady, wide-based, stiff-legged walk. Supportive criteria are not required for a diagnosis of Rett syndrome but may occur in some individuals. In addition, these symptoms, which vary in severity from child to child, may not be observed in very young children but may develop with age. A child with supportive criteria but none of the essential criteria does not have Rett syndrome. Supportive criteria include scoliosis, teeth-grinding, small cold hands and feet in relation to height, abnormal sleep patterns, abnormal muscle tone, heart abnormalities, inappropriate laughing or screaming, intense eye communication, and diminished response to pain.

There is no cure for Rett syndrome. Treatment for the disorder is symptomatic, focusing on the management of symptoms, and supportive, requiring a multidisciplinary approach. Medication may be needed for breathing irregularities and motor difficulties, and anticonvulsant drugs may be used to control seizures.

Accordingly, there remains a need for effective treatments of patients with developmental disorders.

### SUMMARY

Methods of treating a developmental disorder described herein include administering a imidazolone derivative or a pharmaceutically acceptable salt thereof to a patient in need thereof to provide improvement in one or more symptoms of the disorder. In embodiments, methods of treating a developmental disorder described herein include administering imepitoin or a pharmaceutically acceptable salt thereof to a patient in need thereof to provide improvement in one or more symptoms of the disorder. In embodiments, methods of treating a developmental disorder described herein include administering ELB-139 or a pharmaceutically acceptable salt thereof to a patient in need thereof to provide improvement in one or more symptoms of the disorder. In embodiments, methods of treating a developmental disorder described herein include administering a imidazolone derivative or a pharmaceutically acceptable salt thereof to a patient in need thereof to provide improvement in next day functioning of the patient. In embodiments, methods of treating a developmental disorder described herein include administering imepitoin or a pharmaceutically acceptable salt thereof to a patient in need thereof to provide improvement in next day functioning of the patient. In embodiments, methods of treating a developmental disorder described herein include administering ELB-139 or a pharmaceutically acceptable salt thereof to a patient in need thereof to provide improvement in next day functioning of the patient.

In embodiments, the developmental disorder may be an Autistic Spectrum Disorder, pervasive developmental disorder, Autism, Angelman syndrome, Fragile X syndrome, Fragile X-associated tremor/ataxia syndrome (FXTAS), Rett syndrome, Asperger's syndrome, Childhood Disintegrative Disorder, Attention-deficit/hyperactivity disorder (ADHD), Landau-Kleffner Syndrome, Prader-Willi Syndrome, Rasmussen's syndrome, Dravet syndrome, Tardive Dyskinesia, seizure disorder and/or Williams Syndrome. In embodiments, the developmental disorder may be a seizure disorder such as epilepsy, epilepsy with generalized tonic-clonic seizures, epilepsy with myoclonic absences, frontal lobe epilepsy, temporal lobe epilepsy, infantile spasms (West syndrome), childhood absence epilepsy, juvenile myoclonic epilepsy (JME), vaccine-related encephalopathy, intractable childhood epilepsy (ICE), essential tremor, acute repetitive seizures, benign rolandic epilepsy, status epilepticus, refractory status, epilepticus, super-refractory status epilepticus (SRSE), PCDH19 pediatric epilepsy, increased seizure activity (also called serial or cluster seizures), or breakthrough seizures. In embodiments, the developmental disorder may be a seizure disorder such as Doose syndrome, CDKL5 disorder, West's syndrome, Lennox-Gastaut syndrome (LGS) and Ohtahara syndrome. In embodiments, the seizure disorder is associated with a sodium channel protein type 1 subunit alpha (Scn1a)-related disorder. In embodiments, the seizure disorder may either be associated with, or independent of, any of the above-listed developmental disorders.

### DETAILED DESCRIPTION

Described herein are methods of treating a developmental disorder that include administering a imidazolone derivative to a patient in need thereof. In embodiments, the imidazolone derivative can be represented by the compound of formula 1.

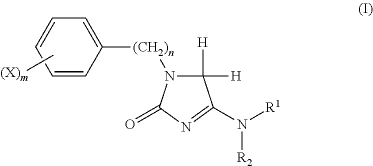

wherein X is hydrogen, C₁₋₄-alkyl, C₁₋₄ alkoxy, trifluoromethyl, or halogen, R¹ and R² are independently C₁₋₄-alkyl, cycloalkyl or heteroalkyl residue, or R¹ and R² together form C₂₋₆ alkylene in which a —CH₂-group is optionally replaced by oxygen, nitrogen or sulfur, or a 4-methylpiperazino group, and n is 0 or 1, and m is 0, 1, 2, 3, 4 or 5.

Examples of compounds of formula 1 are 1-phenyl-4-morpholino-imidazolin-2-one, 1-(4-methoxy)-4-piperidino-imidazolin-2-one, 1-(4-chlorophenyl)-4-morpholino-imidazolin-2-one (imepitoin), 1-(4-chlorophenyl)-4-piperidino-imidazolin-2-one (ELB139), 1-(4-chlorophenyl)-4-dimethylamino-imidazolin-2-one, 1-(4-bromophenyl)-4-morpholino-imidazolin-2-one, 1-(3-chlorophenyl)-4-morpholino-imidazolin-2-one, 1-(4-chlorophenyl)-4-hexamethyleneimino-imidazolin-2-one, 1-(4-chlorophenyl)-4-(4-methylpiperazino) imidazoline-2-one, 1-(4-methylphenyl)-4-morpholino-imidazolin-2-one, 1-(4-chlorophenyl)-4-(cyclohexyl-methylamino)-imidazoline-2-one, and 1-(4-fluorophenyl)-4-morpholino-imidazolin-2-one, 1-benzyl-4-morpholino-imidazolin-2-one. Imepitoin has the following structure and may also be referred to by the following names:

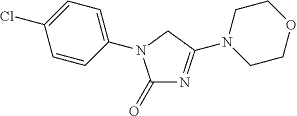 [SMILES: O=C1N=C(N2CCOCC2)CN1c1ccc(Cl)cc1]

3-(4-chlorophenyl)-5-morpholin-4-yl-4H-imidazol-2-one, 2H-Imidazol-2-one, 1-(4-chlorophenyl)-1,5-dihydro-4-(4-morpholinyl), (1-(4-chlorophenyl)-4-(4-morpholinyl)-2,5-dihydro-1H-imidazol-2-one), (1-(4-chlorophenyl)-4-morpholino-imidazolin-2-one, AWD 131-138 or ELB138.

ELB139 has the following structure and may also referred to by the following names:

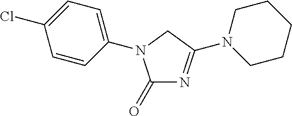 [SMILES: O=C1N=C(N2CCCCC2)CN1c1ccc(Cl)cc1]

1-(4-chlorophenyl)-4-piperidinoimidazolin-2-one, 3-(4-chlorophenyl)-5-piperidin-1-yl-4H-imidazol-2-one or AWD 131-139.

In embodiments, the developmental disorder may be an Autistic Spectrum Disorder, pervasive developmental disorder, Autism, Angelman syndrome, Fragile X syndrome, Fragile X-associated tremor/ataxia syndrome (FXTAS), Rett syndrome, Asperger's syndrome, Childhood Disintegrative Disorder, Attention-deficit/hyperactivity disorder (ADHD), Landau-Kleffner Syndrome, Prader-Willi Syndrome, Rasmussen's syndrome, Dravet syndrome, Tardive Dyskinesia, seizure disorder and/or Williams Syndrome. In embodiments, the developmental disorder may be a seizure disorder such as epilepsy, epilepsy with generalized tonic-clonic seizures, epilepsy with myoclonic absences, frontal lobe epilepsy, temporal lobe epilepsy, infantile spasms (West syndrome), childhood absence epilepsy, juvenile myoclonic epilepsy (JME), vaccine-related encephalopathy, intractable childhood epilepsy (ICE), essential tremor, acute repetitive seizures, benign rolandic epilepsy, status epilepticus, refractory status, epilepticus, super-refractory status epilepticus (SRSE), PCDH19 pediatric epilepsy, increased seizure activity (also called serial or cluster seizures), or breakthrough seizures. In embodiments, the developmental disorder may be a seizure disorder such as Doose syndrome, CDKL5 disorder, West's syndrome, Lennox-Gastaut syndrome (LGS) and Ohtahara syndrome. In embodiments, the seizure disorder is associated with a sodium channel protein type 1 subunit alpha (Scn1a)-related disorder. In embodiments, the seizure disorder may either be associated with, or independent of, any of the above-listed developmental disorders.

In embodiments, provided are methods and compositions for treating a developmental syndrome by administering to a subject in need thereof a pharmaceutical composition including an effective amount of a compound according to formula 1 or a pharmaceutically acceptable salt thereof. In embodiments, provided are methods and compositions for treating a developmental syndrome by administering to a subject in need thereof a pharmaceutical composition including an effective amount of 1-phenyl-4-morpholino-imidazolin-2-one, 1-(4-methoxy)-4-piperidino-imidazolin-2-one, 1-(4-chlorophenyl)-4-morpholino-imidazolin-2-one (imepitoin), I-(4-chlorophenyl)-4-piperidino-imidazolin-2-one (ELB139), 1-(4-chlorophenyl)-4-dimethylamino-imidazolin-2-one, 1-(4-bromophenyl)-4-morpholine-imidazolin-2-one, 1-(3-chlorophenyl)-4-morpholino-imidazolin-2-one, 1-(4-chlorophenyl)-4-hexamethyleneimino-imidazolin-2-one, 1-(4-chlorophenyl)-4-(4-methylpiperazino) imidazoline-2-one, 1-(4-methylphenyl)-4-morpholino-imidazolin-2-one, 1-(4-chlorophenyl)-4-(cyclohexyl-methylamino)-imidazoline-2-one, 1-(4-fluorophenyl)-4-morpholino-imidazolin-2-one, 1-benzyl-4-morpholino-imidazolin-2-one or a pharmaceutically acceptable salt of any of the foregoing. In embodiments, provided are methods and compositions for treating a developmental syndrome by administering to a subject in need thereof a pharmaceutical composition including an effective amount of imepitoin or a pharmaceutically acceptable salt thereof. In embodiments, provided are methods and compositions for treating a developmental syndrome by administering to a subject in need thereof a pharmaceutical composition including an effective amount of ELB139 or a pharmaceutically acceptable salt thereof.

Methods of treating a developmental disorder described herein include administering a compound according to formula 1 or a pharmaceutically acceptable salt thereof to a patient in need thereof to provide improvement in one or more symptoms of the disorder. In embodiments, methods of treating a developmental disorder described herein include administering 1-phenyl-4-morpholino-imidazolin-2-one, 1-(4-methoxy) piperidino-imidazolin-2-one, 1-(4-chlorophenyl)-4-morpholino-imidazolin-2-one (imepitoin), 1-(4-chlorophenyl)-4-piperidino-imidazolin-2-one (ELB139), 1-(4-chlorophenyl)-4-dimethylamino-imidazolin-2-one, 1-(4-bromophenyl)-4-morpholino-imidazolin-2-one, 1-(3-chlorophenyl)-4-morpholino-imidazolin-2-one, 1-(4-chlorophenyl)-4-hexamethyleneimino-imidazolin-2-one, 1-(4-chlorophenyl)-4-(4-methylpiperazino) imidazoline-2-one, 1-(4-methylphenyl)-4-morpholino-imidazolin-2-one, 1-(4-chlorophenyl)-4-(cyclohexyl-methylamino)-imidazoline-2-one, 1-(4-fluorophenyl)-4-imidazolin-2-one, 1-benzyl-4-morpholino-imidazolin-2-one or a pharmaceutically acceptable salt of any of the foregoing to a patient in need thereof to provide improvement in one or more symptoms of the disorder. In embodiments, methods of treating a developmental disorder described herein include administering imepitoin or a pharmaceutically acceptable salt thereof to a patient in need thereof to provide improvement in one or more symptoms of the disorder. In embodiments, methods of treating a developmental disorder described herein include administering ELB139 or a pharmaceutically acceptable salt thereof to a patient in need thereof to provide improvement in one or more symptoms of the disorder.

Symptoms may include, but are not limited to, ataxia, gait, speech impairment, vocalization, frequent repetition of words or phrases, monotonous speech, social awkwardness, cognition, motor activity, clinical seizure, hypotonia, hypertonia, feeding difficulty, drooling, mouthing behavior, sleep difficulties, hand flapping, rocking, easily provoked laughter, short attention span, reduced sensation, numbness or tingling, pain, or muscle weakness in the lower limbs, inability to control the bladder or bowel, chronic pain syndromes, such as fibromyalgia and chronic migraine, hypothyroidism, hypertension, insomnia, sleep apnea, vertigo, olfactory dysfunction and hearing loss. In embodiments, provided in accordance with the present disclosure is improvement in cognition. Cognition refers to the mental processes involved in gaining knowledge and comprehension, such as thinking, knowing, remembering, judging, and problem solving. These higher-level functions of the brain encompass language, imagination, perception, and the planning and execution of complex behaviors.

In embodiments, the methods described herein are effective to reduce, delay, or prevent one or more symptoms of a developmental disorder. For example, the effect of a composition including a compound of formula 1 or a pharmaceutically acceptable salt thereof, e.g., imepitoin or ELB139, or a pharmaceutically acceptable salt of either imepitoin or ELB139, on a particular symptom, pharmacologic, or physiologic indicator can be compared to an untreated subject, or the condition of the subject prior to treatment. In embodiments, the symptom, pharmacologic, and/or physiologic indicator is measured in a subject prior to treatment, and again one or more times after treatment is initiated. In embodiments, the control is a reference level, or average determined based on measuring the symptom, pharmacologic, or physiologic indicator in one or more subjects that do not have the disease or condition to be treated (e.g., healthy subjects). In embodiments, the effect of the treatment is compared to a conventional treatment that is known the art.

In embodiments, provided herein are methods of treating a developmental disorder including administering to a patient in need thereof a pharmaceutical composition including a compound according to formula 1 or a pharmaceutically acceptable salt thereof, e.g., imepitoin or ELB139, or a pharmaceutically acceptable salt of either imepitoin or ELB139, wherein the composition provides improvement of at least one symptom for more than 4 hours after administration of the pharmaceutical composition to the patient. In embodiments, the improvement of at least one symptom for more than 6 hours after administration of the pharmaceutical composition to the patient is provided in accordance with the present disclosure. In embodiments, improvement in at least one symptom for at least e.g., 8 hours, 10 hours, 12 hours, 15 hours, 18 hours, 20 hours, or 24 hours after administration of the pharmaceutical composition to the patient is provided in accordance with the present disclosure. In embodiments, improvement in at least one symptom for 12 hours after administration of the pharmaceutical composition to the patient is provided in accordance with the present disclosure.

In embodiments, methods of treating a developmental disorder described herein include administering a imidazolone derivative or a pharmaceutically acceptable salt thereof to a patient in need thereof to provide improvement in next day functioning of the patient. In embodiments, methods of treating a developmental disorder described herein include administering a compound of formula 1 or a pharmaceutically acceptable salt thereof to a patient in need thereof to provide improvement in next day functioning of the patient. In embodiments, methods of treating a developmental disorder described herein include administering 1-phenyl-4-morpholino-imidazolin-2-one, 1-(4-methoxy)-4-piperidino-imidazolin-2-one, 1-(4-chlorophenyl)-4-morpholino-imidazolin-2-one (imepitoin), 1-(4-chlorophenyl)-4-piperidino-imidazolin-2-one (ELB139), 1-(4-chlorophenyl)-4-dimethylamino-imidazolin-2-one, 1-(4-bromophenyl)-4-morpholino-imidazolin-2-one, 1-(3-chlorophenyl)-4-morpholino-imidazolin-2-one, 1-(4-chlorophenyl)-4-hexamethyleneimino-imidazolin-2-one, 1-(4-chlorophenyl)-4-(4-methylpiperazino) imidazoline-2-one, 1-(4-methylphenyl)-4-morpholino-imidazolin-2-one, 1-(4-chlorophenyl)-4-(cyclohexyl-methylamino)-imidazoline-2-one, 1-(4-fluorophenyl)-4-morpholino-imidazolin-2-one, 1-benzyl-4-morpholino-imidazolin-2-one or a pharmaceutically acceptable salt of any of the foregoing to a patient in need thereof to provide improvement in next day functioning of the patient. In embodiments, methods of treating a developmental disorder described herein include administering imepitoin or a pharmaceutically acceptable salt thereof to a patient in need thereof to provide improvement in next day functioning of the patient. In embodiments, methods of treating a developmental disorder described herein include administering ELB139 or a pharmaceutically acceptable salt thereof to a patient in need thereof to provide improvement in next day functioning of the patient. “Improvement in next day functioning” or “wherein there is improvement in next day functioning” refers to improvement wherein after passage of a night, the beneficial effect on at least one symptom lasts over a period of time, e.g., 4 hours, 6 hours, 12 hours, 24 hours. etc.

In embodiments, provided herein are methods of treating a developmental disorder including administering to a patient in need thereof a compound of formula 1, such as imepitoin or ELB 139, or a pharmaceutically acceptable salt of either, which provides an in vivo plasma profile, wherein the in vivo plasma profile of the patient 10 hours after administration of the compound according to formula 1 or a pharmaceutically acceptable salt thereof is reduced by more than 50% and the method provides improvement in the patient for more than 10, 12, 14, 16, 18, 20, 22 or 24 hours after administration. In embodiments, provided herein are methods of treating a developmental disorder including administering to a patient in need thereof the compound according to formula 1, such as imepitoin or ELB 139, or a pharmaceutically acceptable salt of either, which provides an in vivo plasma profile, wherein the in vivo plasma profile of the patient 10 hours after administration of the compound according to formula 1, such as imepitoin or ELB 139, or a pharmaceutically acceptable salt of either imepitoin or ELB 139, is reduced by more than 55% and the method provides improvement in the patient for more than 10, 12, 14, 16, 18, 20, 22 or 24 hours after administration. In embodiments, provided herein are methods of treating a developmental disorder including administering to a patient in need thereof a compound according to formula 1, such as imepitoin or ELB 139, or a pharmaceutically acceptable salt of either imepitoin or ELB 139, which provides an in vivo plasma profile, wherein the in vivo plasma profile of the patient 10 hours after administration of the compound according to formula 1, such as imepitoin or ELB 139, or a pharmaceutically acceptable salt of either imepitoin or ELB 139, is reduced by more than 60% and the method provides improvement in the patient for more than 10, 12, 14, 16, 18, 20, 22 or 24 hours after administration. In embodiments, provided herein are methods of treating a developmental disorder including administering to a patient in need thereof a compound according to formula 1, such as imepitoin or ELB 139, or a pharmaceutically acceptable salt of either imepitoin or ELB 139, which provides an in vivo plasma profile, wherein the in vivo plasma profile of the patient 10 hours after administration of the compound according to formula 1, such as imepitoin or ELB 139, or a pharmaceutically acceptable salt of either imepitoin or ELB 139, is reduced by more than 65% and the method provides improvement in the patient for more than 6, 8, 10, 12, 14, 16, 18, 20, 22 or 24 hours after administration. In embodiments, provided herein are methods of treating a developmental disorder including administering to a patient in need thereof a compound according to formula 1, such as imepitoin or ELB 139, or a pharmaceutically acceptable salt of either imepitoin or ELB 139, which provides an in vivo plasma profile, wherein the in vivo plasma profile of the patient 10 hours after administration of the compound according to formula 1, such as imepitoin or ELB 139, or a pharmaceutically acceptable salt of either imepitoin or ELB 139, is reduced by more than 70% and the method provides improvement in the patient for more than 6, 8, 10, 12, 14, 16, 18, 20, 22 or 24 hours after administration.

In embodiments, methods of treating a developmental disorder include administering to a patient in need thereof about 10 mg to about 5000 mg of a compound of formula 1 or a pharmaceutically acceptable salt thereof. In embodiments, about 10 mg to about 5000 mg of a compound of formula 1 or a pharmaceutically acceptable salt thereof is administered in 24 hours. In embodiments, a compound of formula 1 or a pharmaceutically acceptable salt thereof is administered in divided doses over 24 hours.

In embodiments, the patient is administered 10 mg to 20 mg, 20 mg to 30 mg, 30 mg to 40 mg, 50 mg to 75 mg, 75 mg to 100 mg, 100 mg to 125 mg, 125 mg to 150 mg, 150 mg to 175 mg, 175 mg to 200 mg, 200 mg to 225 mg, 225 mg to 250 mg, 250 mg to 275 mg, 275 mg to 300 mg, 300 mg, to 325 mg, 325 mg to 350 mg, 350 mg to 375 mg, 375 mg to 400 mg, 400 mg to 425 mg, 425 mg to 450 mg, 450 mg to 475 mg, 475 mg to 500 mg, 500 mg to 525 mg, 525 mg to 550 mg, 550 mg to 575 mg, 575 mg to 600 mg, 600 mg to 625 mg, 625 mg to 650 mg, 650 mg to 675 mg, 675 mg to 700 mg, 700 mg to 725 mg, 725 mg to 750 mg, 750 mg to 775 mg, 775 mg to 800 mg, 800 mg to 825 mg, 825 mg to 850 mg, 850 mg to 875 mg, 875 mg to 900 mg, 900 mg to 925 mg, 925 mg to 950 mg, 950 mg to 975 mg, 975 mg to 1000 mg, 1000 mg to 1025 mg, 1025 mg to 1050 mg, 1050 mg to 1075 mg, 1075 mg to 1100 mg, 1100 mg to 1125 mg, 1125 mg to 1150 mg, 1150 mg to 1175 mg, 1175 mg to 1200 mg, 1200 mg to 1225 mg, 1225 mg to 1250 mg, 1250 mg to 1275 mg, 1275 mg to 1300 mg, 1300 mg to 1325 mg, 1325 mg to 1350 mg, 1350 mg to 1375 mg, 1375 mg to 1400 mg, 1400 mg to 1425 mg, 1425 mg to 1450 mg, 1450 mg to 1475 mg, 1475 mg to 1500 mg, 1500 mg to 1525 mg, 1525 mg to 1550 mg, 1550 mg to 1575 mg, 1575 mg to 1600 mg, 1600 mg to 1625 mg, 1625 mg to 1650 mg, 1650 mg to 1675 mg, 1675 mg to 1700 mg, 1700 mg to 1725 mg, 1725 mg to 1750 mg, 1750 mg to 1775 mg, 1775 mg to 1800 mg, 1800 mg to 1825 mg, 1825 mg to 1850 mg, 1850 mg to 1875 mg, 1875 mg to 1900 mg, 1900 mg to 1925 mg, 1925 mg to 1950 mg, 1950 mg to 1975 mg, 1975 mg to 2000 mg, 2000 mg to 2025 mg, 2025 mg to 2050 mg, 2050 mg to 2075 mg, 2075 mg to 2100 mg, 2100 mg to 2125 mg, 2125 mg to 2150 mg, 2150 mg to 2175 mg, 2175 mg to 2200 mg, 2200 mg to 2225 mg, 2225 mg to 2250 mg, 2250 mg to 2275 mg, 2275 mg to 2300 mg, 2300 mg to 2325 mg, 2325 mg to 2350 mg, 2350 mg to 2375 mg, 2375 mg to 2400 mg, 2400 mg to 2425 mg, 2425 mg to 2450 mg, 2450 mg to 2475 mg, 2475 mg to 2500 mg, 2500 mg to 2525 mg, 2525 mg to 2550 mg, 2550 mg to 2575 mg, 2575 mg to 2600 mg, 2600 mg to 2625 mg, 2625 mg to 2650 mg, 2650 mg to 2675 mg, 2675 mg to 2700 mg, 2700 mg to 2725 mg, 2725 mg to 2750 mg, 2750 mg to 2775 mg, 2775 mg to 2800 mg, 2800 mg to 2825 mg, 2825 mg to 2850 mg, 2850 mg to 2875 mg, 2875 mg to 2900 mg, 2900 mg to 2925 mg, 2925 mg to 2950 mg, 2950 mg to 2975 mg, or 2975 mg to 3000 mg, 3000 mg to 3025 mg, 3025 mg to 3050 mg, 3050 mg to 3075 mg, 3075 mg to 3100 mg, 3100 mg to 3125 mg, 3125 mg to 3150 mg, 3150 mg to 3175 mg, 3175 mg to 3200 mg, 3200 mg to 3225 mg, 3225 mg to 3250 mg, 3250 mg to 3275 mg, 3275 mg to 3300 mg, 3300 mg to 3325 mg, 3325 mg to 3350 mg, 3350 mg to 3375 mg, 3375 mg to 3400 mg, 3400 mg to 3425 mg, 3425 mg to 3450 mg, 3450 mg to 3475 mg, 3475 mg to 3500 mg, 3500 mg to 3525 mg, 3525 mg to 3550 mg, 3550 mg to 3575 mg, 3575 mg to 3600 mg, 3600 mg to 3625 mg, 3625 mg to 3650 mg, 3650 mg to 3675 mg, 3675 mg to 3700 mg, 3700 mg to 3725 mg, 3725 mg to 3750 mg, 3750 mg to 3775 mg, 3775 mg to 3800 mg, 3800 mg to 3825 mg, 3825 mg to 3850 mg, 3850 mg to 3875 mg, 3875 mg to 3900 mg, 3900 mg to 3925 mg, 3925 mg to 3950 mg, 3950 mg to 3975 mg, 3975 mg to 4000 mg, 4000 mg to 4025 mg, 4025 mg to 4050 mg, 4050 mg to 4075 mg, 4075 mg to 4100 mg, 4100 mg to 4125 mg, 4125 mg to 4150 mg, 4150 mg to 4175 mg, 4175 mg to 4200 mg, 4200 mg to 4225 mg, 4225 mg to 4250 mg, 4250 mg to 4275 mg, 4275 mg to 4300 mg, 4300 mg to 4325 mg, 4325 mg to 4350 mg, 4350 mg to 4375 mg, 4375 mg to 4400 mg, 4400 mg to 4425 mg, 4425 mg to 4450 mg, 4450 mg to 4475 mg, 4475 mg to 4500 mg, 4500 mg to 4525 mg, 4525 mg to 4550 mg, 4550 mg to 4575 mg, 4575 mg to 4600 mg, 4600 mg to 4625 mg, 4625 mg to 4650 mg, 4650 mg to 4675 mg, 4675 mg to 4700 mg, 4700 mg to 4725 mg, 4725 mg to 4750 mg, 4750 mg to 4775 mg, 4775 mg to 4800 mg, 4800 mg to 4825 mg, 4825 mg to 4850 mg, 4850 mg to 4875 mg, 4875 mg to 4900 mg, 4900 mg to 4925 mg, 4925 mg to 4950 mg, 4950 mg to 4975 mg, or 4975 mg to 5000 mg, of a compound of formula 1 or a pharmaceutically acceptable salt thereof.

In embodiments, the patient is administered 10 mg, 20 mg, 30 mg, 40 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1225 mg, 1250 mg, 1275 mg, 1300 mg, 1325 mg, 1350 mg, 1375 mg, 1400 mg, 1425 mg, 1450 mg, 1475 mg, 1500 mg, 1525 mg, 1550 mg, 1575 mg, 1600 mg, 1625 mg, 1650 mg, 1675 mg, 1700 mg, 1725 mg, 1750 mg, 1775 mg, 1800 mg, 1825 mg, 1850 mg, 1875 mg, 1900 mg, 1925 mg, 1950 mg, 1975 mg, 2000 mg, 2025 mg, 2250 mg, 2275 mg, 2300 mg, 2325 mg, 2350 mg, 2375 mg, 2400 mg, 2425 mg, 2450 mg, 2475 mg, 2500 mg, 2525 mg, 2550 mg, 2575 mg, 2600 mg, 2625 mg, 2650 mg, 2675 mg, 2700 mg, 2725 mg, 2750 mg, 2775 mg, 2800 mg, 2825 mg, 2850 mg, 2875 mg, 2900 mg, 2925 mg, 2950 mg, 2975 mg, or 3000 mg, 3025 mg, 3250 mg, 3275 mg, 3300 mg, 3325 mg, 3350 mg, 3375 mg, 3400 mg, 3425 mg, 3450 mg, 3475 mg, 3500 mg, 3525 mg, 3550 mg, 3575 mg, 3600 mg, 3625 mg, 3650 mg, 3675 mg, 3700 mg, 3725 mg, 3750 mg, 3775 mg, 3800 mg, 3825 mg, 3850 mg, 3875 mg, 3900 mg, 3925 mg, 3950 mg, 3975 mg, 4000 mg, 4025 mg, 4250 mg, 4275 mg, 4300 mg, 4325 mg, 4350 mg, 4375 mg, 4400 mg, 4425 mg, 4450 mg, 4475 mg, 4500 mg, 4525 mg, 4550 mg, 4575 mg, 4600 mg, 4625 mg, 4650 mg, 4675 mg, 4700 mg, 4725 mg, 4750 mg, 4775 mg, 4800 mg, 4825 mg, 4850 mg, 4875 mg, 4900 mg, 4925 mg, 4950 mg, 4975 mg, or 5000 mg of a compound of formula 1 or a pharmaceutically acceptable salt thereof.

In embodiments methods of treating a developmental disorder include administering to a patient in need thereof a pharmaceutical composition including about 10 mg to about 5000 mg of a compound of formula 1 or a pharmaceutically acceptable salt thereof.

In embodiments, the pharmaceutical compositions include 10 mg to 20 mg, 20 mg to 30 mg, 30 mg to 40 mg, 40 mg to 50 mg, 50 mg to 75 mg, 75 mg to 100 mg, 100 mg to 125 mg, 125 mg to 150 mg, 150 mg to 175 mg, 175 mg to 200 mg, 200 mg to 225 mg, 225 mg to 250 mg, 250 mg to 275 mg, 275 mg to 300 mg, 300 mg to 325 mg, 325 mg to 350 mg, 350 mg to 375 mg, 375 mg to 400 mg, 400 mg to 425 mg, 425 mg to 450 mg, 450 mg to 475 mg, 475 mg to 500 mg, 500 mg to 525 mg, 525 mg to 550 mg, 550 mg to 575 mg, 575 mg to 600 mg, 600 mg to 625 mg, 625 mg to 650 mg, 650 mg to 675 mg, 675 mg to 700 mg, 700 mg to 725 mg, 725 mg to 750 mg, 750 mg to 775 mg, 775 mg to 800 mg, 800 mg to 825 mg, 825 mg to 850 mg, 850 mg to 875 mg, 875 mg to 900 mg, 900 mg to 925 mg, 925 mg to 950 mg, 950 mg to 975 mg, 975 mg to 1000 mg, 1000 mg to 1025 mg, 1025 mg to 1050 mg, 1050 mg to 1075 mg, 1075 mg to 1100 mg, 1100 mg to 1125 mg, 1125 mg to 1150 mg, 1150 mg to 1175 mg, 1175 mg to 1200 mg, 1200 mg to 1225 mg, 1225 mg to 1250 mg, 1250 mg to 1275 mg, 1275 mg to 1300 mg, 1300 mg to 1325 mg, 1325 mg to 1350 mg, 1350 mg to 1375 mg, 1375 mg to 1400 mg, 1400 mg to 1425 mg, 1425 mg to 1450 mg, 1450 mg to 1475 mg, 1475 mg to 1500 mg, 1500 mg to 1525 mg, 1525 mg to 1550 mg, 1550 mg to 1575 mg, 1575 mg to 1600 mg, 1600 mg to 1625 mg, 1625 mg to 1650 mg, 1650 mg to 1675 mg, 1675 mg to 1700 mg, 1700 mg to 1725 mg, 1725 mg to 1750 mg, 1750 mg to 1775 mg, 1775 mg to 1800 mg, 1800 mg to 1825 mg, 1825 mg to 1850 mg, 1850 mg to 1875 mg, 1875 mg to 1900 mg, 1900 mg to 1925 mg, 1925 mg to 1950 mg, 1950 mg to 1975 mg, 1975 mg to 2000 mg, 2000 mg to 2025 mg, 2025 mg to 2050 mg, 2050 mg to 2075 mg, 2075 mg to 2100 mg, 2100 mg to 2125 mg, 2125 mg to 2150 mg, 2150 mg to 2175 mg, 2175 mg to 2200 mg, 2200 mg to 2225 mg, 2225 mg to 2250 mg, 2250 mg to 2275 mg, 2275 mg to 2300 mg, 2300 mg to 2325 mg, 2325 mg to 2350 mg, 2350 mg to 2375 mg, 2375 mg to 2400 mg, 2400 mg to 2425 mg, 2425 mg to 2450 mg, 2450 mg to 2475 mg, 2475 mg to 2500 mg, 2500 mg to 2525 mg, 2525 mg to 2550 mg, 2550 mg to 2575 mg, 2575 mg to 2600 mg, 2600 mg to 2625 mg, 2625 mg to 2650 mg, 2650 mg to 2675 mg, 2675 mg to 2700 mg, 2700 mg to 2725 mg, 2725 mg to 2750 mg, 2750 mg to 2775 mg, 2775 mg to 2800 mg, 2800 mg to 2825 mg, 2825 mg to 2850 mg, 2850 mg to 2875 mg, 2875 mg to 2900 mg, 2900 mg to 2925 mg, 2925 mg to 2950 mg, 2950 mg to 2975 mg, 2975 mg to 3000 mg, of a compound of formula 1 or a pharmaceutically acceptable salt thereof.

In embodiments, the pharmaceutical compositions include 10 mg, 20 mg, 30 mg, 40 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1225 mg, 1250 mg, 1275 mg, 1300 mg, 1325 mg, 1350 mg, 1375 mg, 1400 mg, 1425 mg, 1450 mg, 1475 mg, 1500 mg, 1525 mg, 1550 mg, 1575 mg, 1600 mg, 1625 mg, 1650 mg, 1675 mg, 1700 mg, 1725 mg, 1750 mg, 1775 mg, 1800 mg, 1825 mg, 1850 mg, 1875 mg, 1900 mg, 1925 mg, 1950 mg, 1975 mg, 2000 mg, 2025 mg, 2250 mg, 2275 mg, 2300 mg, 2325 mg, 2350 mg, 2375 mg, 2400 mg, 2425 mg, 2450 mg, 2475 mg, 2500 mg, 2525 mg, 2550 mg, 2575 mg, 2600 mg, 2625 mg, 2650 mg, 2675 mg, 2700 mg, 2725 mg, 2750 mg, 2775 mg, 2800 mg, 2825 mg, 2850 mg, 2875 mg, 2900 mg, 2925 mg, 2950 mg, 2975 mg, 3000 mg, of a compound of formula 1 or a pharmaceutically acceptable salt thereof. In embodiments, a compound of formula 1 or a pharmaceutically acceptable salt thereof is administered once, twice, three or four times a day.

In embodiments, methods of treating a developmental disorder include administering to a patient in need thereof about 10 mg to about 3000 mg of imepitoin or a pharmaceutically acceptable salt thereof. In embodiments, about 10 mg to about 3000 mg of imepitoin or a pharmaceutically acceptable salt thereof is administered in 24 hours. In embodiments, imepitoin or a pharmaceutically acceptable salt thereof is administered in divided doses over 24 hours. In embodiments, imepitoin or a pharmaceutically acceptable salt thereof is administered once, twice, three or four times a day. For example, 200 mg three times daily, 300 mg three times daily, 400 mg three times daily, or 500 mg three times daily.

In embodiments, the patient is administered 10 mg to 20 mg, 20 mg to 30 mg, 30 mg to 40 mg, 50 mg to 75 mg, 75 mg to 100 mg, 100 mg to 125 mg, 125 mg to 150 mg, 150 mg to 175 mg, 175 mg to 200 mg, 200 mg to 225 mg, 225 mg to 250 mg, 250 mg to 275 mg, 275 mg to 300 mg, 300 mg, to 325 mg, 325 mg to 350 mg, 350 mg to 375 mg, 375 mg to 400 mg, 400 mg to 425 mg, 425 mg to 450 mg, 450 mg to 475 mg, 475 mg to 500 mg, 500 mg to 525 mg, 525 mg to 550 mg, 550 mg to 575 mg, 575 mg to 600 mg, 600 mg to 625 mg, 625 mg to 650 mg, 650 mg to 675 mg, 675 mg to 700 mg, 700 mg to 725 mg, 725 mg to 750 mg, 750 mg to 775 mg, 775 mg to 800 mg, 800 mg to 825 mg, 825 mg to 850 mg, 850 mg to 875 mg, 875 mg to 900 mg, 900 mg to 925 mg, 925 mg to 950 mg, 950 mg to 975 mg, 975 mg to 1000 mg, 1000 mg to 1025 mg, 1025 mg to 1050 mg, 1050 mg to 1075 mg, 1075 mg to 1100 mg, 1100 mg to 1125 mg, 1125 mg to 1150 mg, 1150 mg to 1175 mg, 1175 mg to 1200 mg, 1200 mg to 1225 mg, 1225 mg to 1250 mg, 1250 mg to 1275 mg, 1275 mg to 1300 mg, 1300 mg to 1325 mg, 1325 mg to 1350 mg, 1350 mg to 1375 mg, 1375 mg to 1400 mg, 1400 mg to 1425 mg, 1425 mg to 1450 mg, 1450 mg to 1475 mg, 1475 mg to 1500 mg, 1500 mg to 1525 mg, 1525 mg to 1550 mg, 1550 mg to 1575 mg, 1575 mg to 1600 mg, 1600 mg to 1625 mg, 1625 mg to 1650 mg, 1650 mg to 1675 mg, 1675 mg to 1700 mg, 1700 mg to 1725 mg, 1725 mg to 1750 mg, 1750 mg to 1775 mg, 1775 mg to 1800 mg, 1800 mg to 1825 mg, 1825 mg to 1850 mg, 1850 mg to 1875 mg, 1875 mg to 1900 mg, 1900 mg to 1925 mg, 1925 mg to 1950 mg, 1950 mg to 1975 mg, 1975 mg to 2000 mg, 2000 mg to 2025 mg, 2025 mg to 2050 mg, 2050 mg to 2075 mg, 2075 mg to 2100 mg, 2100 mg to 2125 mg, 2125 mg to 2150 mg, 2150 mg to 2175 mg, 2175 mg to 2200 mg, 2200 mg to 2225 mg, 2225 mg to 2250 mg, 2250 mg to 2275 mg, 2275 mg to 2300 mg, 2300 mg to 2325 mg, 2325 mg to 2350 mg, 2350 mg to 2375 mg, 2375 mg to 2400 mg, 2400 mg to 2425 mg, 2425 mg to 2450 mg, 2450 mg to 2475 mg, 2475 mg to 2500 mg, 2500 mg to 2525 mg, 2525 mg to 2550 mg, 2550 mg to 2575 mg, 2575 mg to 2600 mg, 2600 mg to 2625 mg, 2625 mg to 2650 mg, 2650 mg to 2675 mg, 2675 mg to 2700 mg, 2700 mg to 2725 mg, 2725 mg to 2750 mg, 2750 mg to 2775 mg, 2775 mg to 2800 mg, 2800 mg to 2825 mg, 2825 mg to 2850 mg, 2850 mg to 2875 mg, 2875 mg to 2900 mg, 2900 mg to 2925 mg, 2925 mg to 2950 mg, 2950 mg to 2975 mg, or 2975 mg to 3000 mg, 3000 mg to 3025 mg, 3025 mg to 3050 mg, 3050 mg to 3075 mg, 3075 mg to 3100 mg, 3100 mg to 3125 mg, 3125 mg to 3150 mg, 3150 mg to 3175 mg, 3175 mg to 3200 mg, 3200 mg to 3225 mg, 3225 mg to 3250 mg, 3250 mg to 3275 mg, 3275 mg to 3300 mg, 3300 mg to 3325 mg, 3325 mg to 3350 mg, 3350 mg to 3375 mg, 3375 mg to 3400 mg, 3400 mg to 3425 mg, 3425 mg to 3450 mg, 3450 mg to 3475 mg, 3475 mg to 3500 mg, 3500 mg to 3525 mg, 3525 mg to 3550 mg, 3550 mg to 3575 mg, 3575 mg to 3600 mg, 3600 mg to 3625 mg, 3625 mg to 3650 mg, 3650 mg to 3675 mg, 3675 mg to 3700 mg, 3700 mg to 3725 mg, 3725 mg to 3750 mg, 3750 mg to 3775 mg, 3775 mg to 3800 mg, 3800 mg to 3825 mg, 3825 mg to 3850 mg, 3850 mg to 3875 mg, 3875 mg to 3900 mg, 3900 mg to 3925 mg, 3925 mg to 3950 mg, 3950 mg to 3975 mg, or 3975 mg to 4000 mg, 4000 mg to 4025 mg, 4025 mg to 4050 mg, 4050 mg to 4075 mg, 4075 mg to 4100 mg, 4100 mg to 4125 mg, 4125 mg to 4150 mg, 4150 mg to 4175 mg, 4175 mg to 4200 mg, 4200 mg to 4225 mg, 4225 mg to 4250 mg, 4250 mg to 4275 mg, 4275 mg to 4300 mg, 4300 mg to 4325 mg, 4325 mg to 4350 mg, 4350 mg to 4375 mg, 4375 mg to 4400 mg, 4400 mg to 4425 mg, 4425 mg to 4450 mg, 4450 mg to 4475 mg, 4475 mg to 4500 mg, 4500 mg to 4525 mg, 4525 mg to 4550 mg, 4550 mg to 4575 mg, 4575 mg to 4600 mg, 4600 mg to 4625 mg, 4625 mg to 4650 mg, 4650 mg to 4675 mg, 4675 mg to 4700 mg, 4700 mg to 4725 mg, 4725 mg to 4750 mg, 4750 mg to 4775 mg, 4775 mg to 4800 mg, 4800 mg to 4825 mg, 4825 mg to 4850 mg, 4850 mg to 4875 mg, 4875 mg to 4900 mg, 4900 mg to 4925 mg, 4925 mg to 4950 mg, 4950 mg to 4975 mg, or 4975 mg to 5000 mg, of imepitoin or a pharmaceutically acceptable salt thereof.

In embodiments, the patient is administered 10 mg, 20 mg, 30 mg, 40 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1225 mg, 1250 mg, 1275 mg, 1300 mg, 1325 mg, 1350 mg, 1375 mg, 1400 mg, 1425 mg, 1450 mg, 1475 mg, 1500 mg, 1525 mg, 1550 mg, 1575 mg, 1600 mg, 1625 mg, 1650 mg, 1675 mg, 1700 mg, 1725 mg, 1750 mg, 1775 mg, 1800 mg, 1825 mg, 1850 mg, 1875 mg, 1900 mg, 1925 mg, 1950 mg, 1975 mg, 2000 mg, 2025 mg, 2250 mg, 2275 mg, 2300 mg, 2325 mg, 2350 mg, 2375 mg, 2400 mg, 2425 mg, 2450 mg, 2475 mg, 2500 mg, 2525 mg, 2550 mg, 2575 mg, 2600 mg, 2625 mg, 2650 mg, 2675 mg, 2700 mg, 2725 mg, 2750 mg, 2775 mg, 2800 mg, 2825 mg, 2850 mg, 2875 mg, 2900 mg, 2925 mg, 2950 mg, 2975 mg, or 3000 mg, 3025 mg, 3250 mg, 3275 mg, 3300 mg, 3325 mg, 3350 mg, 3375 mg, 3400 mg, 3425 mg, 3450 mg, 3475 mg, 3500 mg, 3525 mg, 3550 mg, 3575 mg, 3600 mg, 3625 mg, 3650 mg, 3675 mg, 3700 mg, 3725 mg, 3750 mg, 3775 mg, 3800 mg, 3825 mg, 3850 mg, 3875 mg, 3900 mg, 3925 mg, 3950 mg, 3975 mg, 4000 mg, 4025 mg, 4250 mg, 4275 mg, 4300 mg, 4325 mg, 4350 mg, 4375 mg, 4400 mg, 4425 mg, 4450 mg, 4475 mg, 4500 mg, 4525 mg, 4550 mg, 4575 mg, 4600 mg, 4625 mg, 4650 mg, 4675 mg, 4700 mg, 4725 mg, 4750 mg, 4775 mg, 4800 mg, 4825 mg, 4850 mg, 4875 mg, 4900 mg, 4925 mg, 4950 mg, 4975 mg, or 5000 mg of imepitoin or a pharmaceutically acceptable salt thereof

In embodiments methods of treating a developmental disorder include administering to a patient in need thereof a pharmaceutical composition including about 10 mg to about 3000 mg of imepitoin or a pharmaceutically acceptable salt thereof.

In embodiments, the pharmaceutical compositions include 10 mg to 20 mg, 20 mg to 30 mg, 30 mg to 40 mg, 40 mg to 50 mg, 50 mg to 75 mg, 75 mg to 100 mg, 100 mg to 125 mg, 125 mg to 150 mg, 150 mg to 175 mg, 175 mg to 200 mg, 200 mg to 225 mg, 225 mg to 250 mg, 250 mg to 275 mg, 275 mg to 300 mg, 300 mg to 325 mg, 325 mg to 350 mg, 350 mg to 375 mg, 375 mg to 400 mg, 400 mg to 425 mg, 425 mg to 450 mg, 450 mg to 475 mg, 475 mg to 500 mg, 500 mg to 525 mg, 525 mg to 550 mg, 550 mg to 575 mg, 575 mg to 600 mg, 600 mg to 625 mg, 625 mg to 650 mg, 650 mg to 675 mg, 675 mg to 700 mg, 700 mg to 725 mg, 725 mg to 750 mg, 750 mg to 775 mg, 775 mg to 800 mg, 800 mg to 825 mg, 825 mg to 850 mg, 850 mg to 875 mg, 875 mg to 900 mg, 900 mg to 925 mg, 925 mg to 950 mg, 950 mg to 975 mg, 975 mg to 1000 mg, 1000 mg to 1025 mg, 1025 mg to 1050 mg, 1050 mg to 1075 mg, 1075 mg to 1100 mg, 1100 mg to 1125 mg, 1125 mg to 1150 mg, 1150 mg to 1175 mg, 1175 mg to 1200 mg, 1200 mg to 1225 mg, 1225 mg to 1250 mg, 1250 mg to 1275 mg, 1275 mg to 1300 mg, 1300 mg to 1325 mg, 1325 mg to 1350 mg, 1350 mg to 1375 mg, 1375 mg to 1400 mg, 1400 mg to 1425 mg, 1425 mg to 1450 mg, 1450 mg to 1475 mg, 1475 mg to 1500 mg, 1500 mg to 1525 mg, 1525 mg to 1550 mg, 1550 mg to 1575 mg, 1575 mg to 1600 mg, 1600 mg to 1625 mg, 1625 mg to 1650 mg, 1650 mg to 1675 mg, 1675 mg to 1700 mg, 1700 mg to 1725 mg, 1725 mg to 1750 mg, 1750 mg to 1775 mg, 1775 mg to 1800 mg, 1800 mg to 1825 mg, 1825 mg to 1850 mg, 1850 mg to 1875 mg, 1875 mg to 1900 mg, 1900 mg to 1925 mg, 1925 mg to 1950 mg, 1950 mg to 1975 mg, 1975 mg to 2000 mg, 2000 mg to 2025 mg, 2025 mg to 2050 mg, 2050 mg to 2075 mg, 2075 mg to 2100 mg, 2100 mg to 2125 mg, 2125 mg to 2150 mg, 2150 mg to 2175 mg, 2175 mg to 2200 mg, 2200 mg to 2225 mg, 2225 mg to 2250 mg, 2250 mg to 2275 mg, 2275 mg to 2300 mg, 2300 mg to 2325 mg, 2325 mg to 2350 mg, 2350 mg to 2375 mg, 2375 mg to 2400 mg, 2400 mg to 2425 mg, 2425 mg to 2450 mg, 2450 mg to 2475 mg, 2475 mg to 2500 mg, 2500 mg to 2525 mg, 2525 mg to 2550 mg, 2550 mg to 2575 mg, 2575 mg to 2600 mg, 2600 mg to 2625 mg, 2625 mg to 2650 mg, 2650 mg to 2675 mg, 2675 mg to 2700 mg, 2700 mg to 2725 mg, 2725 mg to 2750 mg, 2750 mg to 2775 mg, 2775 mg to 2800 mg, 2800 mg to 2825 mg, 2825 mg to 2850 mg, 2850 mg to 2875 mg, 2875 mg to 2900 mg, 2900 mg to 2925 mg, 2925 mg to 2950 mg, 2950 mg to 2975 mg, 2975 mg to 3000 mg, of imepitoin or a pharmaceutically acceptable salt thereof.

In embodiments, the pharmaceutical compositions include 10 mg, 20 mg, 30 mg, 40 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1225 mg, 1250 mg, 1275 mg, 1300 mg, 1325 mg, 1350 mg, 1375 mg, 1400 mg, 1425 mg, 1450 mg, 1475 mg, 1500 mg, 1525 mg, 1550 mg, 1575 mg, 1600 mg, 1625 mg, 1650 mg, 1675 mg, 1700 mg, 1725 mg, 1750 mg, 1775 mg, 1800 mg, 1825 mg, 1850 mg, 1875 mg, 1900 mg, 1925 mg, 1950 mg, 1975 mg, 2000 mg, 2025 mg, 2250 mg, 2275 mg, 2300 mg, 2325 mg, 2350 mg, 2375 mg, 2400 mg, 2425 mg, 2450 mg, 2475 mg, 2500 mg, 2525 mg, 2550 mg, 2575 mg, 2600 mg, 2625 mg, 2650 mg, 2675 mg, 2700 mg, 2725 mg, 2750 mg, 2775 mg, 2800 mg, 2825 mg, 2850 mg, 2875 mg, 2900 mg, 2925 mg, 2950 mg, 2975 mg, 3000 mg, of imepitoin or a pharmaceutically acceptable salt thereof.

In embodiments, methods of treating a developmental disorder include administering to a patient in need thereof about 10 mg to about 5000 mg of ELB139 or a pharmaceutically acceptable salt thereof. In embodiments, about 10 mg to about 5000 mg of ELB139 or a pharmaceutically acceptable salt thereof is administered in 24 hours. In embodiments, ELB139 or a pharmaceutically acceptable salt thereof is administered in divided doses over 24 hours. In embodiments, ELB139 or a pharmaceutically acceptable salt thereof is administered once, twice, three or four times a day. For example, 200 mg three times daily, 300 mg three times daily, 400 mg three times daily, or 500 mg three times daily.

In embodiments, the patient is administered 10 mg to 20 mg, 20 mg to 30 mg, 30 mg to 40 mg, 50 mg to 75 mg, 75 mg to 100 mg, 100 mg to 125 mg, 125 mg to 150 mg, 150 mg to 175 mg, 175 mg to 200 mg, 200 mg to 225 mg, 225 mg to 250 mg, 250 mg to 275 mg, 275 mg to 300 mg, 300 mg, to 325 mg, 325 mg to 350 mg, 350 mg to 375 mg, 375 mg to 400 mg, 400 mg to 425 mg, 425 mg to 450 mg, 450 mg to 475 mg, 475 mg to 500 mg, 500 mg to 525 mg, 525 mg to 550 mg, 550 mg to 575 mg, 575 mg to 600 mg, 600 mg to 625 mg, 625 mg to 650 mg, 650 mg to 675 mg, 675 mg to 700 mg, 700 mg to 725 mg, 725 mg to 750 mg, 750 mg to 775 mg, 775 mg to 800 mg, 800 mg to 825 mg, 825 mg to 850 mg, 850 mg to 875 mg, 875 mg to 900 mg, 900 mg to 925 mg, 925 mg to 950 mg, 950 mg to 975 mg, 975 mg to 1000 mg, 1000 mg to 1025 mg, 1025 mg to 1050 mg, 1050 mg to 1075 mg, 1075 mg to 1100 mg, 1100 mg to 1125 mg, 1125 mg to 1150 mg, 1150 mg to 1175 mg, 1175 mg to 1200 mg, 1200 mg to 1225 mg, 1225 mg to 1250 mg, 1250 mg to 1275 mg, 1275 mg to 1300 mg, 1300 mg to 1325 mg, 1325 mg to 1350 mg, 1350 mg to 1375 mg, 1375 mg to 1400 mg, 1400 mg to 1425 mg, 1425 mg to 1450 mg, 1450 mg to 1475 mg, 1475 mg to 1500 mg, 1500 mg to 1525 mg, 1525 mg to 1550 mg, 1550 mg to 1575 mg, 1575 mg to 1600 mg, 1600 mg to 1625 mg, 1625 mg to 1650 mg, 1650 mg to 1675 mg, 1675 mg to 1700 mg, 1700 mg to 1725 mg, 1725 mg to 1750 mg, 1750 mg to 1775 mg, 1775 mg to 1800 mg, 1800 mg to 1825 mg, 1825 mg to 1850 mg, 1850 mg to 1875 mg, 1875 mg to 1900 mg, 1900 mg to 1925 mg, 1925 mg to 1950 mg, 1950 mg to 1975 mg, 1975 mg to 2000 mg, 2000 mg to 2025 mg, 2025 mg to 2050 mg, 2050 mg to 2075 mg, 2075 mg to 2100 mg, 2100 mg to 2125 mg, 2125 mg to 2150 mg, 2150 mg to 2175 mg, 2175 mg to 2200 mg, 2200 mg to 2225 mg, 2225 mg to 2250 mg, 2250 mg to 2275 mg, 2275 mg to 2300 mg, 2300 mg to 2325 mg, 2325 mg to 2350 mg, 2350 mg to 2375 mg, 2375 mg to 2400 mg, 2400 mg to 2425 mg, 2425 mg to 2450 mg, 2450 mg to 2475 mg, 2475 mg to 2500 mg, 2500 mg to 2525 mg, 2525 mg to 2550 mg, 2550 mg to 2575 mg, 2575 mg to 2600 mg, 2600 mg to 2625 mg, 2625 mg to 2650 mg, 2650 mg to 2675 mg, 2675 mg to 2700 mg, 2700 mg to 2725 mg, 2725 mg to 2750 mg, 2750 mg to 2775 mg, 2775 mg to 2800 mg, 2800 mg to 2825 mg, 2825 mg to 2850 mg, 2850 mg to 2875 mg, 2875 mg to 2900 mg, 2900 mg to 2925 mg, 2925 mg to 2950 mg, 2950 mg to 2975 mg, or 2975 mg to 3000 mg, 3000 mg to 3025 mg, 3025 mg to 3050 mg, 3050 mg to 3075 mg, 3075 mg to 3100 mg, 3100 mg to 3125 mg, 3125 mg to 3150 mg, 3150 mg to 3175 mg, 3175 mg to 3200 mg, 3200 mg to 3225 mg, 3225 mg to 3250 mg, 3250 mg to 3275 mg, 3275 mg to 3300 mg, 3300 mg to 3325 mg, 3325 mg to 3350 mg, 3350 mg to 3375 mg, 3375 mg to 3400 mg, 3400 mg to 3425 mg, 3425 mg to 3450 mg, 3450 mg to 3475 mg, 3475 mg to 3500 mg, 3500 mg to 3525 mg, 3525 mg to 3550 mg, 3550 mg to 3575 mg, 3575 mg to 3600 mg, 3600 mg to 3625 mg, 3625 mg to 3650 mg, 3650 mg to 3675 mg, 3675 mg to 3700 mg, 3700 mg to 3725 mg, 3725 mg to 3750 mg, 3750 mg to 3775 mg, 3775 mg to 3800 mg, 3800 mg to 3825 mg, 3825 mg to 3850 mg, 3850 mg to 3875 mg, 3875 mg to 3900 mg, 3900 mg to 3925 mg, 3925 mg to 3950 mg, 3950 mg to 3975 mg, or 3975 mg to 4000 mg, 4000 mg to 4025 mg, 4025 mg to 4050 mg, 4050 mg to 4075 mg, 4075 mg to 4100 mg, 4100 mg to 4125 mg, 4125 mg to 4150 mg, 4150 mg to 4175 mg, 4175 mg to 4200 mg, 4200 mg to 4225 mg, 4225 mg to 4250 mg, 4250 mg to 4275 mg, 4275 mg to 4300 mg, 4300 mg to 4325 mg, 4325 mg to 4350 mg, 4350 mg to 4375 mg, 4375 mg to 4400 mg, 4400 mg to 4425 mg, 4425 mg to 4450 mg, 4450 mg to 4475 mg, 4475 mg to 4500 mg, 4500 mg to 4525 mg, 4525 mg to 4550 mg, 4550 mg to 4575 mg, 4575 mg to 4600 mg, 4600 mg to 4625 mg, 4625 mg to 4650 mg, 4650 mg to 4675 mg, 4675 mg to 4700 mg, 4700 mg to 4725 mg, 4725 mg to 4750 mg, 4750 mg to 4775 mg, 4775 mg to 4800 mg, 4800 mg to 4825 mg, 4825 mg to 4850 mg, 4850 mg to 4875 mg, 4875 mg to 4900 mg, 4900 mg to 4925 mg, 4925 mg to 4950 mg, 4950 mg to 4975 mg, or 4975 mg to 5000 mg, of ELB139 or a pharmaceutically acceptable salt thereof.

In embodiments, the patient is administered 10 mg, 20 mg, 30 mg, 40 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1225 mg, 1250 mg, 1275 mg, 1300 mg, 1325 mg, 1350 mg, 1375 mg, 1400 mg, 1425 mg, 1450 mg, 1475 mg, 1500 mg, 1525 mg, 1550 mg, 1575 mg, 1600 mg, 1625 mg, 1650 mg, 1675 mg, 1700 mg, 1725 mg, 1750 mg, 1775 mg, 1800 mg, 1825 mg, 1850 mg, 1875 mg, 1900 mg, 1925 mg, 1950 mg, 1975 mg, 2000 mg, 2025 mg, 2250 mg, 2275 mg, 2300 mg, 2325 mg, 2350 mg, 2375 mg, 2400 mg, 2425 mg, 2450 mg, 2475 mg, 2500 mg, 2525 mg, 2550 mg, 2575 mg, 2600 mg, 2625 mg, 2650 mg, 2675 mg, 2700 mg, 2725 mg, 2750 mg, 2775 mg, 2800 mg, 2825 mg, 2850 mg, 2875 mg, 2900 mg, 2925 mg, 2950 mg, 2975 mg, or 3000 mg, 3025 mg, 3250 mg, 3275 mg, 3300 mg, 3325 mg, 3350 mg, 3375 mg, 3400 mg, 3425 mg, 3450 mg, 3475 mg, 3500 mg, 3525 mg, 3550 mg, 3575 mg, 3600 mg, 3625 mg, 3650 mg, 3675 mg, 3700 mg, 3725 mg, 3750 mg, 3775 mg, 3800 mg, 3825 mg, 3850 mg, 3875 mg, 3900 mg, 3925 mg, 3950 mg, 3975 mg, 4000 mg, 4025 mg, 4250 mg, 4275 mg, 4300 mg, 4325 mg, 4350 mg, 4375 mg, 4400 mg, 4425 mg, 4450 mg, 4475 mg, 4500 mg, 4525 mg, 4550 mg, 4575 mg, 4600 mg, 4625 mg, 4650 mg, 4675 mg, 4700 mg, 4725 mg, 4750 mg, 4775 mg, 4800 mg, 4825 mg, 4850 mg, 4875 mg, 4900 mg, 4925 mg, 4950 mg, 4975 mg, or 5000 mg of ELB139 or a pharmaceutically acceptable salt thereof.

In embodiments methods of treating a developmental disorder include administering to a patient in need thereof a pharmaceutical composition including about 10 mg to about 5000 mg of ELB139 or a pharmaceutically acceptable salt thereof.

In embodiments, the pharmaceutical compositions include 10 mg to 20 mg, 20 mg to 30 mg, 30 mg to 40 mg, 40 mg to 50 mg, 50 mg to 75 mg, 75 mg to 100 mg, 100 mg to 125 mg, 125 mg to 150 mg, 150 mg to 175 mg, 175 mg to 200 mg, 200 mg to 225 mg, 225 mg to 250 mg, 250 mg to 275 mg, 275 mg to 300 mg, 300 mg to 325 mg, 325 mg to 350 mg, 350 mg to 375 mg, 375 mg to 400 mg, 400 mg to 425 mg, 425 mg to 450 mg, 450 mg to 475 mg, 475 mg to 500 mg, 500 mg to 525 mg, 525 mg to 550 mg, 550 mg to 575 mg, 575 mg to 600 mg, 600 mg to 625 mg, 625 mg to 650 mg, 650 mg to 675 mg, 675 mg to 700 mg, 700 mg to 725 mg, 725 mg to 750 mg, 750 mg to 775 mg, 775 mg to 800 mg, 800 mg to 825 mg, 825 mg to 850 mg, 850 mg to 875 mg, 875 mg to 900 mg, 900 mg to 925 mg, 925 mg to 950 mg, 950 mg to 975 mg, 975 mg to 1000 mg, 1000 mg to 1025 mg, 1025 mg to 1050 mg, 1050 mg to 1075 mg, 1075 mg to 1100 mg, 1100 mg to 1125 mg, 1125 mg to 1150 mg, 1150 mg to 1175 mg, 1175 mg to 1200 mg, 1200 mg to 1225 mg, 1225 mg to 1250 mg, 1250 mg to 1275 mg, 1275 mg to 1300 mg, 1300 mg to 1325 mg, 1325 mg to 1350 mg, 1350 mg to 1375 mg, 1375 mg to 1400 mg, 1400 mg to 1425 mg, 1425 mg to 1450 mg, 1450 mg to 1475 mg, 1475 mg to 1500 mg, 1500 mg to 1525 mg, 1525 mg to 1550 mg, 1550 mg to 1575 mg, 1575 mg to 1600 mg, 1600 mg to 1625 mg, 1625 mg to 1650 mg, 1650 mg to 1675 mg, 1675 mg to 1700 mg, 1700 mg to 1725 mg, 1725 mg to 1750 mg, 1750 mg to 1775 mg, 1775 mg to 1800 mg, 1800 mg to 1825 mg, 1825 mg to 1850 mg, 1850 mg to 1875 mg, 1875 mg to 1900 mg, 1900 mg to 1925 mg, 1925 mg to 1950 mg, 1950 mg to 1975 mg, 1975 mg to 2000 mg, 2000 mg to 2025 mg, 2025 mg to 2050 mg, 2050 mg to 2075 mg, 2075 mg to 2100 mg, 2100 mg to 2125 mg, 2125 mg to 2150 mg, 2150 mg to 2175 mg, 2175 mg to 2200 mg, 2200 mg to 2225 mg, 2225 mg to 2250 mg, 2250 mg to 2275 mg, 2275 mg to 2300 mg, 2300 mg to 2325 mg, 2325 mg to 2350 mg, 2350 mg to 2375 mg, 2375 mg to 2400 mg, 2400 mg to 2425 mg, 2425 mg to 2450 mg, 2450 mg to 2475 mg, 2475 mg to 2500 mg, 2500 mg to 2525 mg, 2525 mg to 2550 mg, 2550 mg to 2575 mg, 2575 mg to 2600 mg, 2600 mg to 2625 mg, 2625 mg to 2650 mg, 2650 mg to 2675 mg, 2675 mg to 2700 mg, 2700 mg to 2725 mg, 2725 mg to 2750 mg, 2750 mg to 2775 mg, 2775 mg to 2800 mg, 2800 mg to 2825 mg, 2825 mg to 2850 mg, 2850 mg to 2875 mg, 2875 mg to 2900 mg, 2900 mg to 2925 mg, 2925 mg to 2950 mg, 2950 mg to 2975 mg, 2975 mg to 3000 mg, of ELB139 or a pharmaceutically acceptable salt thereof.

In embodiments, the pharmaceutical compositions include 10 mg, 20 mg, 30 mg, 40 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1225 mg, 1250 mg, 1275 mg, 1300 mg, 1325 mg, 1350 mg, 1375 mg, 1400 mg, 1425 mg, 1450 mg, 1475 mg, 1500 mg, 1525 mg, 1550 mg, 1575 mg, 1600 mg, 1625 mg, 1650 mg, 1675 mg, 1700 mg, 1725 mg, 1750 mg, 1775 mg, 1800 mg, 1825 mg, 1850 mg, 1875 mg, 1900 mg, 1925 mg, 1950 mg, 1975 mg, 2000 mg, 2025 mg, 2250 mg, 2275 mg, 2300 mg, 2325 mg, 2350 mg, 2375 mg, 2400 mg, 2425 mg, 2450 mg, 2475 mg, 2500 mg, 2525 mg, 2550 mg, 2575 mg, 2600 mg, 2625 mg, 2650 mg, 2675 mg, 2700 mg, 2725 mg, 2750 mg, 2775 mg, 2800 mg, 2825 mg, 2850 mg, 2875 mg, 2900 mg, 2925 mg, 2950 mg, 2975 mg, 3000 mg, 3025 mg, 3250 mg, 3275 mg, 3300 mg, 3325 mg, 3350 mg, 3375 mg, 3400 mg, 3425 mg, 3450 mg, 3475 mg, 3500 mg, 3525 mg, 3550 mg, 3575 mg, 3600 mg, 3625 mg, 3650 mg, 3675 mg, 3700 mg, 3725 mg, 3750 mg, 3775 mg, 3800 mg, 3825 mg, 3850 mg, 3875 mg, 3900 mg, 3925 mg, 3950 mg, 3975 mg, 4000 mg, 4025 mg, 4250 mg, 4275 mg, 4300 mg, 4325 mg, 4350 mg, 4375 mg, 4400 mg, 4425 mg, 4450 mg, 4475 mg, 4500 mg, 4525 mg, 4550 mg, 4575 mg, 4600 mg, 4625 mg, 4650 mg, 4675 mg, 4700 mg, 4725 mg, 4750 mg, 4775 mg, 4800 mg, 4825 mg, 4850 mg, 4875 mg, 4900 mg, 4925 mg, 4950 mg, 4975 mg, or 5000 mg of ELB139 or a pharmaceutically acceptable salt thereof.

Pharmaceutical compositions herein may be provided with conventional release or modified release profiles. In embodiments, pharmaceutical compositions with different drug release profiles may be combined to create a two phase or three-phase release profile. For example, pharmaceutical compositions may be provided with an immediate release and an extended release profile. In embodiments, pharmaceutical compositions may be provided with an extended release and delayed release profile. Such compositions may be provided as pulsatile formulations, multilayer tablets, or capsules containing tablets, beads, granules, etc. Compositions may be prepared using a pharmaceutically acceptable “carrier” composed of materials that are considered safe and effective. The “carrier” includes all components present in the pharmaceutical formulation other than the active ingredient or ingredients. The term “carrier” includes, but is not limited to, diluents, binders, lubricants, disintegrants, fillers, and coating compositions.

In embodiments, the pharmaceutical compositions described herein may be administered once, twice, three times, four times daily, or every other day. In embodiments, a pharmaceutical composition described herein is provided to the patient in the morning. In embodiments, a pharmaceutical composition described herein is provided to the patient in the evening. In embodiments, a pharmaceutical composition described herein is provided to the patient once in the evening and once in the morning. In embodiments, a pharmaceutical composition described herein is provided to the patient once in the morning, once in the afternoon and once in the evening or at night. In embodiments, the total amount of a compound of formula 1 or a pharmaceutically acceptable salt thereof administered to a subject in a 24-hour period is 50 mg to 5000 mg. In embodiments, the total amount of imepitoin or a pharmaceutically acceptable salt thereof administered to a subject in a 24-hour period is 50 mg to 3000 mg. In embodiments, the total amount of imepitoin or a pharmaceutically acceptable salt thereof administered to a subject in a 24-hour period is 100 mg to 2500 mg. In embodiments, the total amount of imepitoin or a pharmaceutically acceptable salt thereof administered to a subject in a 24-hour period is 500 mg, 600 mg, 750 mg, 800 mg, 850 mg, 1000 mg, 1200 mg, 1600 mg, 1800 mg, 2000 mg or 2500 mg. In embodiments, the total amount of imepitoin or a pharmaceutically acceptable salt thereof administered to a subject in a 24-hour period is 1500 mg.

In embodiments, the total amount of ELB139 or a pharmaceutically acceptable salt thereof administered to a subject in a 24-hour period is 50 mg to 5000 mg. In embodiments, the total amount of ELB139 or a pharmaceutically acceptable salt thereof administered to a subject in a 24-hour period is 100 mg to 2500 mg. In embodiments, the total amount of ELB139 or a pharmaceutically acceptable salt thereof administered to a subject in a 24-hour period is 500 mg, 600 mg, 750 mg, 800 mg, 850 mg, 1000 mg, 1200 mg, 1600 mg, 1800 mg, 2000 mg or 2500 mg. In embodiments, the total amount of ELB139 or a pharmaceutically acceptable salt thereof administered to a subject in a 24-hour period is 1800 mg.

As mentioned above, pharmaceutical compositions herein may be provided with conventional release or modified release profiles. Modified release profiles include immediate release, delayed release, or extended release profiles. Conventional (or unmodified) release oral dosage forms such as tablets or capsules typically release medications into the stomach or intestines as the tablet or capsule shell dissolves. The pattern of drug release from modified release (MR) dosage forms is deliberately changed from that of a conventional dosage form to achieve a desired therapeutic objective and/or better patient compliance. Types of MR drug products include orally disintegrating dosage forms (ODDFs) which provide immediate release, extended release dosage forms, delayed release dosage forms (e.g., enteric coated), and pulsatile release dosage forms.

An ODDF is a solid dosage form containing a medicinal substance or active ingredient which disintegrates rapidly, usually within a matter of seconds when placed upon the tongue. The disintegration time for ODDFs generally range from one or two seconds to about a minute. ODDFs are designed to disintegrate or dissolve rapidly on contact with saliva. This mode of administration can be beneficial to people who may have problems swallowing tablets whether it be from physical infirmity or psychiatric in nature. Patients with Angelman syndrome, Fragile X syndrome, Fragile X-associated tremor/ataxia syndrome or Rett syndrome may exhibit such behavior. Examples of ODDFs include orally disintegrating tablets, capsules and rapidly dissolving films and wafers.

Extended release dosage forms (ERDFs) have extended release profiles and are those that allow a reduction in dosing frequency as compared to that presented by a conventional dosage form, e.g., a solution or unmodified release dosage form. ERDFs provide a sustained duration of action of a drug. Suitable formulations which provide extended release profiles are well-known in the art. For example, coated slow release beads or granules (“beads” and “granules” are used interchangeably herein) in which, e.g., a compound according to formula 1 such as imepitoin, ELB139, or a pharmaceutically acceptable salt of either imepitoin or ELB139, is applied to beads, e.g., confectioners nonpareil beads, and then coated with conventional release retarding materials such as waxes, enteric coatings and the like. In embodiments, beads can be formed in which a compound according to formula 1 such as imepitoin, ELB139, or a pharmaceutically acceptable salt of either imepitoin or ELB139 is mixed with a material to provide a mass from which the drug leaches out. In embodiments, the beads may be engineered to provide different rates of release by varying characteristics of the coating or mass, e.g., thickness, porosity, using different materials, etc. Beads having different rates of release may be combined into a single dosage form to provide variable or continuous release. The beads can be contained in capsules or compressed into tablets. In embodiments, extended release imepitoin dosage forms may contain 500 mg, 750 mg, 1000 mg, 1500 mg, 2000 mg or 2500 mg imepitoin or ELB139 as the active ingredient. In embodiments, a compound of formula 1 such as imepitoin, ELB139, or a pharmaceutically acceptable salt of either imepitoin or ELB139, extended release dosage forms incorporate a dual hydrophilic polymer matrix system. In embodiments, a compound of formula 1 such as imepitoin, ELB139, or a pharmaceutically acceptable salt of either imepitoin or ELB139, is combined with a drug release controlling polymer to form an “inner” phase, which is then incorporated as discrete particles into an “external” phase of a second polymer. After administration, fluid from the gastrointestinal (GI) tract enters the tablet, causing the polymers to hydrate and swell. Drug is released slowly from the dosage form by a process of diffusion through the gel matrix that is essentially independent of pH.

In embodiments, modified dosage forms herein incorporate delayed release dosage forms having delayed release profiles. Delayed release dosage forms can include delayed release tablets or delayed release capsules. A delayed release tablet is a solid dosage form which releases a drug (or drugs) such as a compound according to formula 1 such as imepitoin, ELB139, or a pharmaceutically acceptable salt of either imepitoin or ELB139, at a time other than promptly after administration. A delayed release capsule is a solid dosage form in which the drug is enclosed within either a hard or soft soluble container made from a suitable form of gelatin, and which releases a drug (or drugs) at a time other than promptly after administration. For example, enteric-coated tablets, capsules, particles and beads are well-known examples of delayed release dosage forms. Enteric coated tablets, capsules and particles and beads pass through the stomach and release the drug in the intestine. In embodiments, a delayed release tablet is a solid dosage form containing a conglomerate of medicinal particles that releases a drug (or drugs) at a time other than promptly after administration. In embodiments, the conglomerate of medicinal particles are covered with a coating which delays release of the drug. In embodiments, a delayed release capsule is a solid dosage form containing a conglomerate of medicinal particles that releases a drug (or drugs) at a time other than promptly after administration. In embodiments, the conglomerate of medicinal particles are covered with a coating which delays release of the drug.

Delayed release dosage forms are known to those skilled in the art. For example, coated delayed release beads or granules in which, e.g., as a compound according to formula 1 such as imepitoin, ELB139, or a pharmaceutically acceptable salt of either imepitoin or ELB139, is applied to beads, e.g., confectioners nonpareil beads, and then coated with conventional release delaying materials such as waxes, enteric coatings and the like. In embodiments, beads can be formed in which as a compound according to formula 1 such as imepitoin, ELB139, or a pharmaceutically acceptable salt of either imepitoin or ELB139, is mixed with a material to provide a mass from which the drug leaches out. In embodiments, the beads may be engineered to provide different rates of release by varying characteristics of the coating or mass, e.g., thickness, porosity, using different materials, etc. In embodiments, enteric coated granules of as a compound according to formula 1 such as imepitoin, ELB139, or a pharmaceutically acceptable salt of either imepitoin or ELB139, can be contained in an enterically coated capsule or tablet which releases the granules in the small intestine. In embodiments, the granules have a coating which remains intact until the coated granules reach at least the ileum and thereafter provide a delayed release of the drug in the colon. Suitable enteric coating materials are well known in the art, e.g., Eudragit® coatings such methacrylic acid and methyl methacrylate polymers and others. The granules can be contained in capsules or compressed into tablets.

In embodiments, a compound of formula 1 such as imepitoin, ELB139, or a pharmaceutically acceptable salt of either imepitoin or ELB139, is incorporated into porous inert carriers that provide delayed release profiles. In embodiments, the porous inert carriers incorporate channels or passages from which the drug diffuses into surrounding fluids. In embodiments, a compound of formula 1 such as imepitoin, ELB139, or a pharmaceutically acceptable salt of either imepitoin or ELB139, is incorporated into an ion-exchange resin to provide a delayed release profile. Delayed action may result from a predetermined rate of release of the drug from the resin when the drug-resin complex contacts gastrointestinal fluids and the ionic constituents dissolved therein. In embodiments, membranes are utilized to control rate of release from drug containing reservoirs. In embodiments, liquid preparations may also be utilized to provide a delayed release profile. For example, a liquid preparation consisting of solid particles dispersed throughout a liquid phase in which the particles are not soluble. The suspension is formulated to allow at least a reduction in dosing frequency as compared to that drug presented as a conventional dosage form (e.g., as a solution or a prompt drug-releasing, conventional solid dosage form). For example, a suspension of ion-exchange resin constituents or microbeads.

Unless defined otherwise, all technical and scientific terms used herein have the same meanings as commonly understood by one of skill in the art to which the disclosure herein belongs.

The term “about” or “approximately” as used herein means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and/or up to 1% of a given value. Alternatively, particularly with respect to biological systems or processes, the term can mean within an order of magnitude, preferably within 5-fold, and more preferably within 2-fold, of a value.

“Improvement” refers to the treatment of a developmental disorder such as Angelman syndrome, Fragile X syndrome, Fragile X-associated tremor/ataxia syndrome or Rett syndrome measured relative to at least one symptom.

“PK” refers to the pharmacokinetic profile. Cₘₐₓ is defined as the highest plasma drug concentration estimated during an experiment (ng/ml). Tₘₐₓ is defined as the time when Cₘₐₓ is estimated (min). AUC_{0-∞} is the total area under the plasma drug concentration-time curve, from drug administration until the drug is eliminated (ng□hr/ml). The area under the curve is governed by clearance. Clearance is defined as the volume of blood or plasma that is totally cleared of its content of drug per unit time (ml/min).

“Treating” or “treatment” refers to alleviating or delaying the appearance of clinical symptoms of a disease or condition in a subject that may be afflicted with or predisposed to the disease or condition, but does not yet experience or display clinical or subclinical symptoms of the disease or condition. In certain embodiments, “treating” or “treatment” may refer to preventing the appearance of clinical symptoms of a disease or condition in a subject that may be afflicted with or predisposed to the disease or condition, but does not yet experience or display clinical or subclinical symptoms of the disease or condition. “Treating” or “treatment” also refers to inhibiting the disease or condition, e.g., arresting or reducing its development or at least one clinical or subclinical symptom thereof. “Treating” or “treatment” further refers to relieving the disease or condition, e.g., causing regression of the disease or condition or at least one of its clinical or subclinical symptoms. The benefit to a subject to be treated may be statistically significant, mathematically significant, or at least perceptible to the subject and/or the physician. Nonetheless, prophylactic (preventive) and therapeutic (curative) treatment are two separate embodiments of the disclosure herein.

“Pharmaceutically acceptable” refers to molecular entities and compositions that are “generally regarded as safe”, e.g., that are physiologically tolerable and do not typically produce an allergic or similar untoward reaction, such as gastric upset and the like, when administered to a human. In embodiments, this term refers to molecular entities and compositions approved by a regulatory agency of the federal or a state government, as the GRAS list under section 204(s) and 409 of the Federal Food, Drug and Cosmetic Act, that is subject to premarket review and approval by the FDA or similar lists, the U.S. Pharmacopeia or another generally recognized pharmacopeia for use in animals, and more particularly in humans.

“Effective amount” or “therapeutically effective amount” means a dosage sufficient to alleviate one or more symptoms of a disorder, disease, or condition being treated, or to otherwise provide a desired pharmacological and/or physiologic effect.

“Patient in need thereof” may include individuals that have been diagnosed with a developmental disorder including, for example, Autism, Angelman's syndrome, Fragile X syndrome, Fragile X-associated tremor/ataxia syndrome (FXTAS), Rett's syndrome and/or seizure disorder. The methods may be provided to any individual including, e.g., wherein the patient is a neonate, infant, a pediatric patient (6 months to 12 years), an adolescent patient (age 12-18 years) or an adult (over 18 years).

### EXAMPLES

The Examples provided herein are included solely for augmenting the disclosure herein and should not be considered to be limiting in any respect.

### Example 1

### Prospective Assessment of Imepitoin for the Treatment of Angelman Syndrome

This study may be used to test the effect of imepitoin on the epilepsy phenotype as well as behavioral and physiological deficits in a mouse model for Angelman syndrome (AS). All tests will be performed in adult animals (8-10) weeks. Three separate tests will be performed to determine the effects imepitoin including:

(Test 1) audiogenically-induced seizure test; (Test 2) a battery of motor tests (or tests with a significant motor component and shown to be affected in AS mice): Rotarod, Hanging Wire, Clasping reflex, Marble burying and forced swim test; and (Test 3) a synaptic plasticity test as measured by long-term potentiation measurements (LTP).

Completion of each test may provide a quantitative result of the effect of imepitoin on this test and is represented as a distinct milestone (M1-M3). For the behavioral and electrophysiological assessment, we will use F1 hybrid (C57B16-129S2PasCrl) AS mice. For each of these tests, groups of wild-type and Ube3am-p+ mice will be treated with vehicle or drug as described below. Seizure susceptibility will be tested by inducing audiogenic seizures in AS mice in the 129S2PasCrl background. Seizures will be determined the day before start of treatment (T0; >90% seizures) and 30-90 minutes after the last treatment (T1). Wild-type littermates are included as control (<5% seizures). Since we test at T0 and T1, there is no need for a distinct untreated AS group. At the end of the epilepsy experiments (Test 1), animals will be perfused with PFA to preserve the brain. The animals from Test 2 (motor tests) that are not used for Test 3 (Ephys experiments) will be used to obtain plasma and brain tissue, which will be snap-frozen. These materials will enable future biochemical, molecular and pharmacological measurements. Treatment schedule and randomization: Wild-type and Ube3am-p+ mice will be treated by IP injection morning and evening. Treatment will be commenced 4 weeks prior to testing and continued during subsequent testing, until the mice are sacrificed. Treatment groups are as follows:

Group 1—WT animals treated with vehicle in AM and vehicle in PM
Group 2—Mutant animals treated with vehicle in AM and vehicle in PM
Group 3—Mutant animals treated with vehicle in AM and imepitoin in PM
Group 4—Mutant animals treated with imepitoin in AM and imepitoin in PM

### Example 2

### Prospective Assessment of the Efficacy of Imepitoin in Patients with Angelman Syndrome

This study is designed to determine whether imepitoin will lead to an improvement in one or more symptoms of Angelman syndrome. Participants are randomized into 6 separate treatment groups (A-F). Inclusion criteria for randomization will require that each participant has been previously diagnosed with Angelman syndrome by clinical evaluation or that the participant is diagnosed with one or more of the major and minor criteria for Angelman syndrome.

Major Criteria include:
Functionally severe developmental delay
Speech impairment; none or minimal words used
Movement or balance disorder
Behavioral uniqueness, frequent laughs/smiling, excitable personality, hand flapping, short attention span

Minor Criteria include:
Deceleration in head circumference growth (post-natal)
Seizures (myoclonic, absence, drop, tonic-clonic)
Abnormal EEG (with patterns suggestive of AS, or hypsarrhythmia)
Sleep disturbance
Attraction to or fascination with water
Drooling

After randomization the participants are placed into 6 separate treatment groups (A-F) and a placebo group. Treatment group A receives 250 mg imepitoin in the morning. Treatment group B receives 500 mg imepitoin in the morning. Treatment group C receives 300 mg imepitoin in the morning and 300 mg imepitoin in the evening. Treatment group D receives 300 mg imepitoin in the morning, 300 mg imepitoin in the afternoon and 300 mg imepitoin before bedtime. Treatment group E receives 500 mg imepitoin in the morning, 500 mg imepitoin in the afternoon and 500 mg imepitoin before bedtime. Treatment group F receives 800 mg imepitoin in the morning and 800 mg imepitoin in the evening. Treatment group G receives placebo morning, afternoon and before bedtime. The initial treatment period is for two weeks. After the two week period, a determination will be made as to continuing treatment for an additional two week period.

Participants are assessed throughout the treatment period to determine whether imepitoin administration leads to an improvement in one or more symptoms of Angelman syndrome. Several behavioral domains; communication, attention, mal adaptive behaviors, and hyper-excitability are assessed. To quantify the communication behavior, participants engage in an unstructured play session to elicit speech and non-verbal communication attempts. Speech attempts by the child are transcribed phonetically and categorized into five different types of vocalizations using the Stark Assessment of Early Vocal Development-Revised (SAEVD-R) (Nathani, Ertmer et al. 2006) which categorizes non-speech and pre-speech sounds (protophones), as well as vowels, consonants and syllables.

Gait abnormalities occur in most cases of Angelman syndrome. Thus, five primary spatiotemporal parameters are analyzed: cadence, gait velocity, stride width, step length and percent stance. For each parameter, a principal component analysis is used to establish a gait index for assessment of the subjects.

In addition, primary outcome measures that may be assessed include changes in raw or standard scores between baseline and after trial completion of:
I. Bayley Scales of Infant and Toddler Development, 3rd edition (or the Mullen Scales of Early Learning in the more developmentally advanced subjects);
II. Vineland Adaptive Behavior Scales, 2nd edition (standard scores only);
III. Preschool Language Scale, 4th edition;
IV. Aberrant Behavior Checklist—Community version; and
V. A change from baseline in the Clinical Global Impressions Severity Scale Score.

Secondary outcome measures may include normalization of the electroencephalogram (EEG) signature when comparing post imepitoin administration results to baseline results.

### Example 3

### Prospective Assessment of the Efficacy of ELB139 in Patients with Angelman Syndrome

This study is designed to determine whether ELB139 will lead to an improvement in one or more symptoms of Angelman syndrome. Participants are randomized into 6 separate treatment groups (A-F). Inclusion criteria for randomization will require that each participant has been previously diagnosed with Angelman syndrome by clinical evaluation or that the participant is diagnosed with one or more of the major and minor criteria for Angelman syndrome.

Major Criteria include:
Functionally severe developmental delay
Speech impairment; none or minimal words used
Movement or balance disorder
Behavioral uniqueness, frequent laughs/smiling, excitable personality, hand flapping, short attention span

Minor Criteria include:
Deceleration in head circumference growth (post-natal)
Seizures (myoclonic, absence, drop, tonic-clonic)
Abnormal EEG (with patterns suggestive of AS, or hypsarrhythmia)
Sleep disturbance
Attraction to or fascination with water
Drooling

After randomization the participants are placed into 6 separate treatment groups (A-F) and a placebo group. Treatment group A receives 500 mg ELB139 in the morning. Treatment group B receives 1000 mg ELB139 in the morning. Treatment group C receives 600 mg ELB139 in the morning and 600 mg ELB139 in the evening. Treatment group D receives 600 mg ELB139 in the morning, 600 mg ELB139 in the afternoon and 600 mg ELB139 before bedtime. Treatment group E receives 750 mg ELB139 in the morning, 750 mg ELB139 in the afternoon and 750 mg ELB139 before bedtime. Treatment group F receives 1000 mg ELB139 in the morning and 1000 mg ELB139 in the evening. Treatment group G receives placebo morning, afternoon and before bedtime. The initial treatment period is for two weeks. After the two week period, a determination will be made as to continuing treatment for an additional two week period.

Participants are assessed throughout the treatment period to determine whether ELB139 administration leads to an improvement in one or more symptoms of Angelman syndrome. Several behavioral domains; communication, attention, maladaptive behaviors, and hyper-excitability are assessed. To quantify the communication behavior, participants engage in an unstructured play session to elicit speech and non-verbal communication attempts. Speech attempts by the child are transcribed phonetically and categorized into five different types of vocalizations using the Stark Assessment of Early Vocal Development-Revised (SAEVD-R) (Nathani, Ertmer et al. 2006) which categorizes non-speech and pre-speech sounds (protophones), as well as vowels, consonants and syllables.

Gait abnormalities occur in most cases of Angelman syndrome. Thus, five primary spatiotemporal parameters are analyzed: cadence, gait velocity, stride width, step length and percent stance. For each parameter, a principal component analysis is used to establish a gait index for assessment of the subjects.

In addition, primary outcome measures that may be assessed include changes in raw or standard scores between baseline and after trial completion of:
VI. Bayley Scales of Infant and Toddler Development, 3rd edition (or the Mullen Scales of Early Learning in the more developmentally advanced subjects);
VII. Vineland Adaptive Behavior Scales, 2nd edition (standard scores only);
VIII. Preschool Language Scale, 4th edition;
IX. Aberrant Behavior Checklist—Community version; and
X. A change from baseline in the Clinical Global Impressions Severity Scale Score.

Secondary outcome measures may include normalization of the electroencephalogram (EEG) signature when comparing post ELB139 administration results to baseline results.

### Example 4

### Prospective Assessment of the Efficacy of Imepitoin in Patients with Fragile X Syndrome

This study is designed to determine whether imepitoin leads to an improvement in one or more symptoms of Fragile X syndrome. Participants are randomized into 6 separate treatment groups (A-F). Inclusion criteria for randomization require patients that have been diagnosed with Fragile X syndrome. For example, patients who are at least moderately ill based on a Clinical Global Impression Severity score of at least 4 and have qualifying scores on the ABC-C and IQ test

After randomization the participants are placed into 6 separate treatment groups (A-F) and a placebo group. Treatment group A receives 250 mg imepitoin in the morning. Treatment group B receives 500 mg imepitoin in the morning. Treatment group C receives 300 mg imepitoin in the morning and 300 mg imepitoin in the evening. Treatment group D receives 300 mg imepitoin in the morning, 300 mg imepitoin in the afternoon and 300 mg imepitoin before bedtime. Treatment group E receives 500 mg imepitoin in the morning, 500 mg imepitoin in the afternoon and 500 mg imepitoin before bedtime. Treatment group F receives 800 mg imepitoin in the morning and 800 mg imepitoin in the evening. Treatment group G receives placebo morning, afternoon and before bedtime. The initial treatment period is for two weeks. After the two week period, a determination will be made as to continuing treatment for an additional two week period.

Participants are assessed throughout the treatment period to determine whether administration of imepitoin leads to an improvement in one or more symptoms of Fragile X syndrome. In particular, patients are assessed using one or more primary and secondary outcome measures. Primary Outcome Measures may include:

Change From Baseline in Behavioral Symptoms of Fragile X Syndrome Using the Aberrant Behavior Checklist-Community Edition (ABC-CFX) Total Score;

Global Improvement of Symptoms in Fragile X Using the Clinical Global Impression-Improvement (CGI-I) Scale;

Change From Baseline in Irritability, Lethargy/Withdrawal, Stereotypic Behavior, Hyperactivity, Inappropriate Speech and Social Avoidance Assessed by the Individual Subscales of the ABC-CFX Scale;

Change From Baseline in Repetitive Behaviors Assessed Using the Repetitive Behavior Scale-Revised (RBS-R) Scores;

Visual Analogue Scale (Behavior); Expressive Vocabulary Test; Vineland Adaptive Behavior Scale-II (VABS-II) Adaptive Behavior Composite Score; and Aberrant Behavior Checklist-Community Edition (ABC-C) Composite Score.

### Example 5

### Prospective Assessment of the Efficacy of ELB139 in Patients with Fragile X Syndrome

This study is designed to determine whether ELB139 leads to an improvement in one or more symptoms of Fragile X syndrome. Participants are randomized into 6 separate treatment groups (A-F). Inclusion criteria for randomization require patients that have been diagnosed with Fragile X syndrome. For example, patients who are at least moderately ill based on a Clinical Global Impression Severity score of at least 4 and have qualifying scores on the ABC-C and IQ test.

After randomization the participants are placed into 6 separate treatment groups (A-F) and a placebo group. Treatment group A receives 500 mg ELB139 in the morning. Treatment group B receives 1000 mg ELB139 in the morning. Treatment group C receives 600 mg ELB139 in the morning and 600 mg ELB139 in the evening. Treatment group D receives 600 mg ELB139 in the morning, 600 mg ELB139 in the afternoon and 600 mg ELB139 before bedtime. Treatment group E receives 750 mg ELB139 in the morning, 750 mg ELB139 in the afternoon and 750 mg ELB139 before bedtime. Treatment group F receives 1000 mg ELB139 in the morning and 1000 mg ELB139 in the evening. Treatment group G receives placebo morning, afternoon and before bedtime. The initial treatment period is for two weeks. After the two week period, a determination will be made as to continuing treatment for an additional two week period.

Participants are assessed throughout the treatment period to determine whether administration of ELB139 leads to an improvement in one or more symptoms of Fragile X syndrome. In particular, patients are assessed using one or more primary and secondary outcome measures. Primary Outcome Measures may include:

Change From Baseline in Behavioral Symptoms of Fragile X Syndrome Using the Aberrant Behavior Checklist-Community Edition (ABC-CFX) Total Score; Global Improvement of Symptoms in Fragile X Using the Clinical Global Impression-Improvement (CGI-I) Scale;

Change From Baseline in Irritability, Lethargy/Withdrawal, Stereotypic Behavior, Hyperactivity, Inappropriate Speech and Social Avoidance Assessed by the Individual Subscales of the ABC-CFX Scale;

Change From Baseline in Repetitive Behaviors Assessed Using the Repetitive Behavior Scale-Revised (RBS-R) Scores;

Visual Analogue Scale (Behavior); Expressive Vocabulary Test; Vineland

Adaptive Behavior Scale-II (VABS-II) Adaptive Behavior Composite Score; and Aberrant Behavior Checklist-Community Edition (ABC-C) Composite Score.

### Example 6

### Prospective Assessment of the Efficacy of Imepitoin in Patients with Rett Syndrome

This study is designed to determine whether imepitoin will lead to an improvement in one or more symptoms of Rett syndrome. Participants are randomized into 6 separate treatment groups (A-F). Inclusion criteria for randomization will require that each participant has been previously diagnosed with Rett syndrome by clinical evaluation or that the participant is diagnosed with one or more of the essential and supportive criteria for Rett syndrome. Genetic testing may also be used to assist in confirming diagnosis of Rett syndrome. Of all cases of clinically diagnosed Rett syndrome, between 80-97% are found to have mutations in the MECP2 gene (a “positive” genetic test).

Essential Criteria include:
a period of normal development until between 6 to 18 months
repetitive hand movements including hand washing, hand wringing and hand clasping
a normal head circumference at birth followed by a slowing of the rate of head growth with age (starting between the time a child is 6 months and 4 years old)
significantly impaired expressive and receptive language
shakiness of the torso, which also may involve the limbs, particularly when the child is upset or agitated
unsteady, wide-based, stiff-legged gait and sometimes toe walking

Supportive Criteria include:
seizures
breathing irregularities such as apnea, hyperventilation and air swallowing
abnormal sleep patterns and irritability
muscle rigidity or spasticity
irritability or agitation
electroencephalogram (EEG) abnormalities
scoliosis (curvature of the spine)
chewing and/or swallowing difficulties
teeth-grinding
decreased body fat and muscle mass
poor circulation of the lower extremities with cold and bluish-red feet and legs
decreased mobility with age

After randomization the participants are placed into 6 separate treatment groups (A-F) and a placebo group. Treatment group A receives 250 mg imepitoin in the morning. Treatment group B receives 500 mg imepitoin in the morning. Treatment group C receives 300 mg imepitoin in the morning and 300 mg imepitoin in the evening. Treatment group D receives 300 mg imepitoin in the morning, 300 mg imepitoin in the afternoon and 300 mg imepitoin before bedtime. Treatment group E receives 500 mg imepitoin in the morning, 500 mg imepitoin in the afternoon and 500 mg imepitoin before bedtime. Treatment group F receives 800 mg imepitoin in the morning and 800 mg imepitoin in the evening. Treatment group G receives placebo morning, afternoon and before bedtime. The initial treatment period is for two weeks. After the two week period, a determination will be made as to continuing treatment for an additional two week period.

Participants are assessed throughout the treatment period to determine whether imepitoin administration leads to an improvement in one or more symptoms of Rett syndrome. Several behavioral domains; communication, attention, maladaptive behaviors, and hyper-excitability are assessed. To quantify the communication behavior, participants engage in an unstructured play session to elicit speech and non-verbal communication attempts. Speech attempts by the child are transcribed phonetically and categorized into five different types of vocalizations using the Stark Assessment of Early Vocal Development-Revised (SAEVD-R) (Nathani, Ertmer et al. 2006) which categorizes non-speech and pre-speech sounds (protophones), as well as vowels, consonants and syllables.

Gait abnormalities occur in many cases of Rett syndrome. Thus, five primary spatiotemporal parameters are analyzed: cadence, gait velocity, stride width, step length and percent stance. For each parameter, a principal component analysis is used to establish a gait index for assessment of the subjects.

In addition, primary outcome measures that may be assessed include changes in raw or standard scores between baseline and after trial completion of:
XI. Bayley Scales of Infant and Toddler Development, 3rd edition (or the Mullen Scales of Early Learning in the more developmentally advanced subjects);
XII. Vineland Adaptive Behavior Scales, 2nd edition (standard scores only);
XIII Preschool Language Scale, 4th edition;
XIV. Aberrant Behavior Checklist—Community version; and
XV. A change from baseline in the Clinical Global Impressions Severity Scale Score.

Secondary outcome measures may include normalization of the electroencephalogram (EEG) signature when comparing post imepitoin administration results to baseline results.

### Example 7

### Prospective Assessment of the Efficacy of ELB139 in Patients with Rett Syndrome

This study is designed to determine whether ELB139 will lead to an improvement in one or more symptoms of Rett syndrome. Participants are randomized into 6 separate treatment groups (A-F). Inclusion criteria for randomization will require that each participant has been previously diagnosed with Rett syndrome by clinical evaluation or that the participant is diagnosed with one or more of the essential and supportive criteria for Rett syndrome. Genetic testing may also be used to assist in confirming diagnosis of Rett syndrome. Of all cases of clinically diagnosed Rett syndrome, between 80-97% are found to have mutations in the MECP2 gene (a “positive” genetic test).

Essential Criteria include:
a period of normal development until between 6 to 18 months
repetitive hand movements including hand washing, hand wringing and hand clasping
a normal head circumference at birth followed by a slowing of the rate of head growth with age (starting between the time a child is 6 months and 4 years old)
significantly impaired expressive and receptive language
shakiness of the torso, which also may involve the limbs, particularly when the child is upset or agitated
unsteady, wide-based, stiff-legged gait and sometimes toe walking

Supportive Criteria include:
seizures
breathing irregularities such as apnea, hyperventilation and air swallowing
abnormal sleep patterns and irritability
muscle rigidity or spasticity
irritability or agitation
electroencephalogram (EEG) abnormalities
scoliosis (curvature of the spine)
chewing and/or swallowing difficulties
teeth-grinding
decreased body fat and muscle mass
poor circulation of the lower extremities with cold and bluish-red feet and legs
decreased mobility with age

After randomization the participants are placed into 6 separate treatment groups (A-F) and a placebo group. Treatment group A receives 500 mg ELB139 in the morning. Treatment group B receives 1000 mg ELB139 in the morning. Treatment group C receives 600 mg ELB139 in the morning and 600 mg ELB139 in the evening. Treatment group D receives 600 mg ELB139 in the morning, 600 mg ELB139 in the afternoon and 600 mg ELB139 before bedtime. Treatment group E receives 750 mg ELB139 in the morning, 750 mg ELB139 in the afternoon and 750 mg ELB139 before bedtime. Treatment group F receives 1000 mg ELB139 in the morning and 1000 mg ELB139 in the evening. Treatment group G receives placebo morning, afternoon and before bedtime. The initial treatment period is for two weeks. After the two week period, a determination will be made as to continuing treatment for an additional two week period.

Participants are assessed throughout the treatment period to determine whether ELB139 administration leads to an improvement in one or more symptoms of Rett syndrome. Several behavioral domains; communication, attention, maladaptive behaviors, and hyper-excitability are assessed. To quantify the communication behavior, participants engage in an unstructured play session to elicit speech and non-verbal communication attempts. Speech attempts by the child are transcribed phonetically and categorized into five different types of vocalizations using the Stark Assessment of Early Vocal Development-Revised (SAEVD-R) (Nathani, Ertmer et al. 2006) which categorizes non-speech and pre-speech sounds (protophones), as well as vowels, consonants and syllables.

Gait abnormalities occur in many cases of Rett syndrome. Thus, five primary spatiotemporal parameters are analyzed: cadence, gait velocity, stride width, step length and percent stance. For each parameter, a principal component analysis is used to establish a gait index for assessment of the subjects.

In addition, primary outcome measures that may be assessed include changes in raw or standard scores between baseline and after trial completion of:
XVI. Bayley Scales of Infant and Toddler Development, 3rd edition (or the Mullen Scales of Early Learning in the more developmentally advanced subjects);
XVII. Vineland Adaptive Behavior Scales, 2nd edition (standard scores only);
XVIII. Preschool Language Scale, 4th edition;
XIX. Aberrant Behavior Checklist—Community version; and
XX. A change from baseline in the Clinical Global Impressions Severity Scale Score.

Secondary outcome measures may include normalization of the electroencephalogram (EEG) signature when comparing post ELB139 administration results to baseline results.

Those skilled in the art will recognize, or be able to ascertain using no more than routine experimentation, many equivalents to the specific embodiments described herein. Such equivalents are intended to be encompassed by the claims.

What is claimed is:

1. A method of treating a developmental disorder comprising administering to a patient in need thereof a therapeutically effective amount of a compound of formula 1 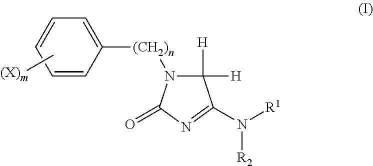
wherein X is hydrogen, C₁₋₄-alkyl, C₁₋₄ alkoxy, trifluoromethyl, or halogen,
R¹ and R² are independently C₁₋₄-alkyl, cycloalkyl or heteroalkyl residue, or
R¹ and R² together form C₂₋₆ alkylene in which a —CH₂-group is optionally replaced by oxygen, nitrogen or sulfur, or a 4-methylpiperazino group, and
n is 0 or 1, and m is 0, 1, 2, 3, 4 or 5; or a pharmaceutically acceptable salt thereof;
wherein the method provides improvement in one or more symptoms of the disorder.

2. The method of claim 1, wherein the developmental disorder is selected from the group consisting of an Autistic Spectrum Disorder, pervasive developmental disorder, Autism, Angelman syndrome, Fragile X syndrome, Fragile X-associated tremor/ataxia syndrome (FXTAS), Rett syndrome, Asperger's syndrome, Childhood Disintegrative Disorder, Landau-Kleffner Syndrome, Prader-Willi Syndrome, Tardive Dyskinesia, seizure disorder and Williams Syndrome.

3. The method of claim 1, wherein the developmental disorder is Angelman syndrome.

4. The method of claim 1, wherein the developmental disorder is Fragile X syndrome.

5. The method of claim 1, wherein the developmental disorder is Fragile X-associated tremor/ataxia syndrome (FXTAS).

6. The method of claim 1, wherein the developmental disorder is Rett syndrome.

7. The method of claim 2, wherein the seizure disorder is epilepsy, epilepsy with generalized tonic-clonic seizures, epilepsy with myoclonic absences, frontal lobe epilepsy, temporal lobe epilepsy, infantile spasms (West syndrome), childhood absence epilepsy, juvenile myoclonic epilepsy (JME), vaccine-related encephalopathy, intractable childhood epilepsy (ICE), essential tremor, acute repetitive seizures, benign rolandic epilepsy, status epilepticus, refractory status, epilepticus, super-refractory status epilepticus (SRSE), PCDH19 pediatric epilepsy, increased seizure activity or breakthrough seizures.

8. The method of claim 2, wherein the seizure disorder is a sodium channel protein type 1 subunit alpha (Scn1a)-related disorder.

9. The method of claim 1, wherein the compound of formula 1 is selected from the group consisting of 1-phenyl-4-morpholino-imidazolin-2-one, 1-(4-methoxy)-4-piperidino-imidazolin-2-one, 1-(1-chlorophenyl)-4-morpholino-imidazolin-2-one (imepitoin), 1-(4-chlorophenyl)-4-piperidino-imidazolin-2-one (ELB139), 1-(4-chlorophenyl)-4-dimethylamino-imidazolin-2-one, 1-(4-bromophenyl)-4-morpholino-imidazolin-2-one, 1-(3-chlorophenyl)-4-morpholino-imidazolin-2-one, 1-(4-chlorophenyl)-4-hexamethyleneimino-imidazolin-2-one, 1-(4-chlorophenyl)-4-(4-methylpiperazino) imidazoline-2-one, 1-(4-methylphenyl)-4-morpholino-imidazolin-2-one, 1-(4-chlorophenyl)-4-(cyclohexyl-methylamino)-imidazoline-2-one, 1-(4-fluorophenyl)-4-morpholino-imidazolin-2-one, 1-benzyl-4-morpholino-imidazolin-2-one and a pharmaceutically acceptable salt of any of the foregoing.

10. The method of claim 1, wherein the compound of formula 1 is imepitoin.

11. The method of claim 1, wherein the compound of formula 1 is ELB139.

12. The method of claim 10, wherein the patient is administered from about 10 mg to about 3000 mg of imepitoin or a pharmaceutically acceptable salt thereof.

13. The method of claim 10, wherein the patient is administered from about 10 mg to about 3000 mg of imepitoin or a pharmaceutically acceptable salt thereof in a 24 hour period.

14. The method of claim 11, wherein the patient is administered from about 10 mg to about 3000 mg of ELB139 or a pharmaceutically acceptable salt thereof.

15. The method of claim 11, wherein the patient is administered from about 10 mg to about 3000 mg of ELB139 or a pharmaceutically acceptable salt thereof in a 24 hour period.

16. The method of claim 1, wherein the in vivo plasma profile of the patient 10 hours after administration of the compound of formula 1 or a pharmaceutically acceptable salt thereof is reduced by more than 50% and the method provides improvement in next day functioning of the patient.

17. The method of claim 1, wherein the in vivo plasma profile of the patient 10 hours after administration of the compound of formula 1 or a pharmaceutically acceptable salt thereof is reduced by more than 50% and the method provides improvement in the patient for more than 10, 12, 14, 16, 18, 20, 22 or 24 hours after administration

18. The method of claim 1, wherein the method provides improvement in at least one symptom selected from the group consisting of ataxia, gait, speech impairment, vocalization, cognition, motor activity, clinical seizure, subclinical seizure, hypotonia, hypertonia, feeding difficulty, drooling, mouthing behavior, sleep difficulties, repetitive hand movements, hand flapping, hand ringing, shakiness of the torso, apnea, hyperventilation and air swallowing, muscle rigidity, spasticity, teeth grinding, poor circulation of the lower extremities, easily provoked laughter and short attention span.

19. The method of claim 1, wherein the method provides improvement in the patient for more than 6 hours.

20. The method of claim 1, wherein the method provides improvement in the patient for more than 8 hours.

21. The method of claim 1, wherein the method provides improvement in the patient for at least 12 hours.

22. The method of claim 1, wherein a composition containing from about 10 mg to about 5000 mg of the compound of formula 1 or a pharmaceutically acceptable salt thereof is administered to the patient.

23. The method of claim 1, wherein a composition containing from about 10 mg to about 3000 mg of imepitoin or a pharmaceutical salt thereof is administered to the patient.

24. The method of claim 23, wherein the composition contains 100 mg, 200 mg, 300 mg, 400 mg, 500 mg, 600 mg, 700 mg, 800 mg, 900 mg, or 1000 mg of imepitoin.

25. The method of claim 1, wherein a composition containing from about 10 mg to about 3000 mg of ELB139 or a pharmaceutical salt thereof is administered to the patient.

26. The method of claim 25, wherein the composition contains 100 mg, 200 mg, 300 mg, 400 mg, 500 mg, 600 mg, 700 mg, 800 mg, 900 mg, 1000 mg, 1100 mg, or 1200 mg of ELB139.

27. The method of claim 22, wherein the composition is an extended release dosage form.

28. The method of claim 22, wherein the composition is a delayed release dosage form.

29. The method of claim 22, wherein the composition is an immediate release dosage form.

30. The method of claim 22, wherein the composition is a conventional release dosage form.

31. A method of treating a developmental disorder comprising administering to a patient in need thereof a therapeutically effective amount of imepitoin or a pharmaceutically acceptable salt thereof wherein the method provides improvement in one or more symptoms of the disorder

32. The method according to claim 31 wherein the disorder is Angelman syndrome.

33. A method of treating a developmental disorder comprising administering to a patient in need thereof a therapeutically effective amount of ELB139 or a pharmaceutically acceptable salt thereof wherein the method provides improvement in one or more symptoms of the disorder

34. The method according to claim 33 wherein the disorder is Angelman syndrome.
